(19) United States
(12) Patent Application Publication
(10) Pub. No.: US 2011/0021943 A1
(43) Pub. Date: Jan. 27, 2011

(54) NEURAL INTERFACE

(75) Inventors: Stephanie Lacour, Cambridge (GB); James Fawcett, Cambridge (GB); James Fitzgerald, Cambridge (GB); Edward Tarte, Birmingham (GB); Steve McMahon, London (GB)

Correspondence Address: McDermott Will & Emery, 600 13th Street, NW, Washington, DC, 20005-3096, US

(73) Assignee: Cambridge Enterprise Limited, Cambridge (GB)

(21) Appl. No.: 12/812,821

(22) PCT Filed: Jan. 16, 2009
(86) PCT No.: PCT/GB09/00123

§ 371 Date: Oct. 12, 2010

(30) Foreign Application Priority Data
Jan. 16, 2008 (GB) 0800797.3

(51) Int. Cl. A61B 5/04; A61N 1/00

(52) U.S. Cl. 600/546; 607/118

(57) ABSTRACT

This invention relates to the neural interface comprising one or more electrode-coupled microchannels. Each electrode-coupled microchannel comprises a microchannel for accommodating regenerating nerve axons; and one or more electrodes exposed to the interior of said microchannel. The one or more electrodes may comprise (i) a stimulation array suitable for generating an extracellular stimulus current which induces an action potential in an axon in the microchannel; and/or (ii) a recording array which detects an extracellular signal in the microchannel indicative of an action potential in an axon in the microchannel. Interfaces as described may be useful in blocking pain, treating misrouted motor nerves; repair or treating nerve injury; or controlling or providing sensation and/or proprioception from a prosthesis.

This invention relates to the neural interfaces, which may for example, be useful in control systems for limb prostheses or for improving the control of a patient's own limb after peripheral nerve repair.

An ideal limb prosthesis is one that the patient can naturally wear and control, i.e. one that is directly interfaced with the patient's peripheral nerve stump. While robotic technology is advanced enough to produce highly sophisticated artificial limbs [1], the technology of interfacing peripheral nerves to electronic prosthetic control systems is still in early development [2]. Alternatively, the patient may have suffered damage to a nerve, with the limb remaining in place. If the nerve is surgically repaired axons will regenerate into the damaged limb, but are not guided back to the correct muscle or sensory structure. A neural interface placed to record signals from the regenerated axons can provide information that can be used to control limb musculature through indwelling stimulators, again providing natural and improved control of the limb. The key factor in determining how much information can be extracted from a neural interface is the number of functioning and independent electrical connections it makes with axons in the nerve to which it is applied. With current devices this is usually far fewer than the number of contact pads it possesses because many of the contacts fail to pick up a signal [3] and some may pick up duplicate signals (crosstalk) [4].

The present inventors have developed a neural interface in which damaged nerve fibres regenerate through an array of electrode-coupled microchannels (‘electroded microchannels’). Confining axons in an electrode coupled micro-channel increases signal levels and reduces crosstalk in detecting action potentials and facilitates fibre type selectivity.

An aspect of the invention provides a neural interface comprising one or more electrode-coupled microchannels,
each electrode-coupled microchannel comprising;
a microchannel for accommodating regenerating nerve axons; and
one or more electrodes exposed to the interior of the microchannel.

The one or more electrodes may, for example, detect extracellular electrical signals in the microchannel when an axon in the microchannel transmits an action potential or generate extracellular electrical signals in the microchannel which stimulate an action potential in the axon.

The regenerating nerve axon in the microchannel may be from a peripheral nerve, optic nerve, cranial nerve, spinal cord or spinal nerve.

The interface may, for example, be positioned at the proximal stump of a damaged nerve to allow axonal regeneration through the microchannels, for example to the distal stump of the damaged nerve.

The microchannels may each accommodate one or more nerve axons of any type. For example, sensory and/or motor axons may be accommodated. The microchannels may further accommodate a blood vessel to supply oxygen and other nutrients to the regenerating nerve axons in the microchannel.

Extracellular electrical signals produced when an axon in the microchannel transmits an action potential may be detected by one or more electrodes exposed to the interior of the microchannel (i.e. a recording array). The one or more electrodes therefore allow information relating to action potentials in the axon to be gathered and analysed. Action potentials from motor and sensory axons may be recorded and analysed in this way. Information gathered from a motor axon may be amplified, recorded and processed and may be used for a range of purposes, for example, to drive a prosthetic limb, to supply information to a muscle stimulator downstream of the site of injury or to study nerve traffic in the regenerated nerve fibre.

One or more electrodes coupled to the microchannel may be useful in transmitting artificial action potentials to an axon in the microchannel by means of capacitive coupling and/or the passage of Faradaic currents between the electrode surface and the axon (i.e. a stimulation array). Artificial action potentials may be produced, for example, in response to input from sensors, such as temperature or pressure sensors, and transmitted via the electrode to a sensory axon in the microchannel, to provide sensory input to the nervous system.

A microchannel is a passage or tunnel in a dielectric material which forms a conduit through which an axon can regenerate or grow. The microchannel is open at both ends to allow the entry and exit of regenerating nerve axons and is closed along its length to surround the regenerating nerve axons within the channel. The microchannel may have any cross-sectional shape. For example, the channel may have a round (e.g. oval or circular) cross-section, or an angular (e.g. square or rectangular) cross-section.

A microchannel suitable for use in an interface described herein may have a diameter or width of at least 1 μm, at least 5 μm, at least 10 μm, at least 20 μm, at least 30 μm, at least 40 μm, at least 50 μm or at least 75 μm. The microchannel may have a diameter or width of up to 1 mm, up to 500 μm, up to 300 μm, up to 200 μm or up to 100 μm. In some preferred embodiments, the microchannel may have a diameter or width of 50 μm to 1 mm, 50 μm to 200 μm, or 40 μm to 100 μm.

A microchannel suitable for use in an interface described herein may have a cross-sectional area of at least 0.002 mm², at least 0.01 mm² or at least 0.05 mm². The microchannel may have a cross-sectional area of up to 0.1 mm², up to 0.5 mm² or up to 1 mm².

A microchannel suitable for use in an interface described herein may have a length of at least 1 mm, at least 1.5 mm, at least 2 mm, at least 3 mm or at least 4 mm. The microchannel may have a length of up to 20 mm, up to 15 mm, or up to 10 mm. In some preferred embodiments, the microchannel may have a length of 1 mm to 10 mm, more preferably 3 mm to 10 mm.

Preferably, the interior of the microchannels provides an environment which promotes and supports growth of one or more of axons, glial cells and blood vessels within the microchannel.

In some embodiments, the microchannels may contain a gel. This provides a structure that encourages axon growth and glial invasion. A gel comprises a matrix of fibres contained in an interstitial electrically conductive medium. Suitable gels may be formed from fibrous materials such as Matrigel™ (BD Biosciences), collagen and spider silk using standard techniques.

In some embodiments, the microchannels may comprise one or more biological factors which promote growth of one or more of axons, glial cells and blood vessels. The biological factors may be incorporated in a gel within the microchannel or attached to the inner walls of the microchannel. Suitable biological factors are described in more detail below.

In use, the microchannels accommodate growing axons in an extracellular fluid which is naturally produced by the surrounding tissue. This fluid provides an ionic conductive medium which contacts the one or more electrodes and allows the detection of action potentials propagating in the axon by the one or more electrodes.

The microchannels may also accommodate glial cells, such as Schwann cells, and blood vessels.

The ratio of the cross-sectional area of microchannels to the cross-sectional area of substrate between the microchannels is generally referred to as transparency. Transparency may encourage the entry of regenerating axons into the microchannels. In some preferred embodiments, an interface may have a transparency of greater than 10%, greater than 20%, greater than 30%, greater than 40%, greater than 50%, or greater than 60%. An interface may have a transparency of up to 70% or up to 80%.

A neural interface may comprise an array of at least 10, at least 30, at least 50, at least 100, or at least 200 electrode-coupled microchannels. In some embodiments, the interface may comprise an array of up to 1000 electrode-coupled microchannels. The interface may additionally comprise one or more microchannels which are not coupled to electrodes.

The microchannels are preferably formed from an electrically insulating (i.e. a dielectric) material. Suitable materials are biocompatible and flexible and may include polymeric materials, such as polyimides, elastomers and gels. Suitable elastomers include silicone.

In some embodiments, a photosensitive polymeric material such as photosensitive polyimide or photopatternable silicone may be employed.

Other suitable insulating materials may include silicon.

In some embodiments, the surface of the microchannel may be coated with a layer of resorbable material. This layer of resorbable material reduces the diameter or cross-sectional area of the microchannel and is progressively resorbed when the interface is in use, for example when implanted in vivo. Progressive resorption of the material leads to a progressive increase in the diameter or cross-sectional area of the microchannel. This allows the microchannel to accommodate increases in the diameter or cross-sectional area of the axons in the microchannel as they regenerate.

Suitable resorbable materials include aliphatic polyesters and co-polyesters such as poly-e-caprolactone (PCL), poly(lactic acid-co-e-caprolactone), biocompatible block polyesterurethanes, polyglycolic acid and polysaccharides (PUs).

The microchannels in the interface are generally associated with one or more electrodes which are in electrical contact with the electrically conductive medium in the microchannel. For example, each microchannel in the interface may be associated with one, two, three or more electrodes. The electrodes do not need to directly contact an axon within the microchannel and the amplitude of the detected signals is not affected by the position of the axon in the microchannel. The amplitude of the detected signal in the microchannels described herein is directly related to the size of the axon. This provides information about the modality of the axon carrying the action potential.

An electrode-coupled microchannel may comprise multiple electrodes, preferably multiple electrodes spaced longitudinally along the microchannel. This may allow the velocity and direction of travel of action potentials in axons within the microchannel to be determined. This may be useful, for example, in distinguishing a sensory neuron from a motor neuron. Other advantageous features of multiple electrode configurations are described below.

The one or more electrodes may be used to detect changes in electrical signal within the medium which are indicative of an action potential within an axon in the microchannel. The electrical signal which is detected by the electrode in the medium may be a change in voltage or current. For recording, typically, the electrodes are 1 Mohm at 1 kHz, although this may be reduced through the use of porous electrode coatings.

Action potentials in the axons may be characterised from the signals detected by the one or more electrodes. For example, the frequency, pattern, and/or amplitude of the action potential may be detected. The direction and velocity of the action potential may be detected in embodiments in which multiple electrodes are longitudinally or axially spaced within the microchannel. In situations where there are several axons in a channel, each may have a distinctive frequency, pattern and size of action potential, allowing the signals from the different axons in the microchannel to be distinguished. Motor and sensory axons in the microchannel may be distinguished by the amplitude and/or direction of the action potential as described below.

In other embodiments, the one or more electrodes may be used to transmit an artificially generated action potential into an axon through the conductive medium by means of capacitive coupling and/or the passage of Faradaic currents between the electrode surface and the axon as described below.

The one or more electrodes are electrically conductive elements which are exposed to the interior of the microchannel so as to form an electrical connection with the conductive extracellular fluid in the microchannel. An electrode may be of any conductive material. In preferred embodiments, the electrode is made of a metallic material, such as gold, platinum or iridium. In other embodiments, the electrode may be made of an organic conductor such as polypyrrole (PPy).

Conveniently, the electrode is in the form of a thin metal film or organic conductor which may be deposited onto the dielectric material, before, after or simultaneous with the formation of the microchannels using known evaporation or sputtering techniques. The electrode lead which connects the electrode to its corresponding electrical pad in the output connector is typically deposited and patterned from the same material at the same time as the electrode.

The electrodes which are exposed to the medium in the microchannel may be treated to its increase surface area. For example, the surface of the pad may be coated with a high porosity compound, such as Platinum Black or Titanium Nitride. Coating the electrode in this way is particularly useful when the electrode is used to stimulate action potentials in the axon.

The data herein shows that action potentials may be induced in axons within microchannels using very low stimulus currents relative to axons outside microchannels. The low amount of current required allows the use of capacitive coupling to stimulate action potentials. In some embodiments, insulated electrodes may be used to supply a stimulus current to the medium by capacitive coupling. For example, the electrodes may be insulated with a dielectric coating to provide capacitive stimulation. Suitable coatings include anodised titanium (Ti—TiO₂), anodised tantalum (Ta—Ta₂O₅), Titanium nitride (TiN) and Barium titanate (BaTiO₃). Coatings may be applied to electrodes by conventional techniques such as anodisation and sputter-coating.

The electrodes may be located anywhere within the microchannel. In some embodiments, electrodes are not positioned adjacent to the ends of the microchannel (e.g. not within L/10 mm, preferably not within L/5 mm, of either end of the microchannel, where L is the total length of the microchannel in mm). In other embodiments, electrodes may be positioned adjacent to the ends of the microchannel. The optimal position for the electrode may be determined by mathematical modelling for particular channel geometries. Particular arrangements of electrodes which are useful for recording arrays and stimulation arrays are described below.

In embodiments in which more than one electrode is coupled to a single microchannel, the electrodes are preferably dispersed axially rather than radially in the microchannel (i.e. longitudinally spaced).

The data herein shows that the position of the electrode relative to the nodes of a myelinated axon does not affect the detection of signal in the extracellular fluid of the microchannel. The electrical signals detected in the extracellular fluid are therefore independent of the positions of the nodes of the nerve axon in the microchannel relative to the electrodes. The electrodes may be positioned in the microchannel independently of the position of the nodes. For example, in some embodiments, the electrode is not positioned at or adjacent a node of a myelinated axon

The interface may further comprise a reference electrode(s) which contacts the conductive medium. The reference electrode may be located within a microchannel or outside the microchannels.

One or more electrodes which detect extracellular signals in the microchannel indicative of action potentials in an axon in the microchannel may be termed a recording array.

In some embodiments, the recording array may be monopolar (i.e. single electrode).

A monopolar recording array may comprise a single electrode which is referenced to ground.

Alternatively, a monopolar recording array may comprise a single electrode which is connected to a differential amplifier circuit. Differential amplifier circuits are well known in the art and provide an amplified signal which represents a multiple of the difference between two input signals.

The differential amplifier circuit may amplify the difference between the signal recorded by the single electrode of the monopolar recording array and a reference signal from another electrode located at the same position (i.e. the mid-point or other longitudinal position) in a different microchannel of the interface. A neural interface as described herein may comprise a first electrode-coupled microchannel and a second electrode-coupled microchannel,
each microchannel having a recording array which comprises a single electrode located at substantially the same position within each microchannel,
the single electrodes from the first and second microchannels being connected to first and second inputs of a differential amplifier circuit to produce a signal indicative of the difference in potential between the first and second microchannels.

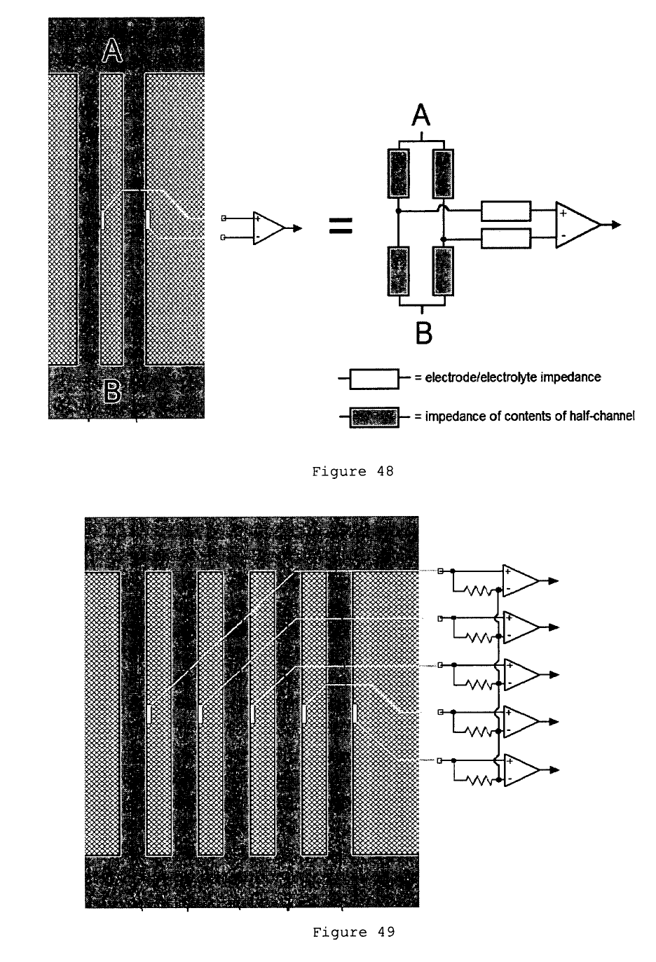

The differential amplification of the signal recorded from single electrodes located in similar positions in two different microchannels may provide improved noise reduction. An example of a monopolar recording array referenced to an electrode in a second microchannel is shown in FIG. 48. Each side of the bridge circuit is formed by the impedances of the two halves of one channel. Since external noise presents as a voltage between A and B (V_{AB}); if R_{AC}=R_{CB} and the electrode impedances are matched the noise voltage V_{AB} is cancelled as a common mode in the output signal from the amplifier.

Alternatively, the differential amplifier circuit may amplify the difference between the signal recorded by the single electrode of the monopolar recording array and a reference signal which is the average signal (i.e. the commoned signal) recorded by single electrodes located at the same position in multiple (i.e. two or more) different microchannels of the interface. A neural interface as described herein may comprise a plurality of electrode-coupled microchannels, preferably three or more electrode-coupled microchannels,
each microchannel being linked to a differential amplifier circuit and having a recording array which comprises a single electrode which is located at the same position within each microchannel and is connected to a first input of the differential amplifier circuit,
wherein the second input of each differential amplifier circuit is connected to common output from all the single electrodes in the plurality of microchannels,
such that the differential amplifier circuit linked to each microchannel produces a signal indicative of the difference in potential between the microchannel and the average potential in the plurality of microchannels.

The differential amplification of the signal recorded from one channel and a reference derived from the signals from a plurality of channels provides improved noise reduction. An example of a monopolar recording array referenced to electrodes in multiple different microchannels is shown in FIG. 49. In this example, the reference side of the bridge circuit for each channel is an average of the mid-channel potentials from multiple channels. As above, if R_{AC}=R_{CB} and the electrode impedances are matched, noise voltage V_{AB} is cancelled as a common mode in the output signal from the amplifier. Using the average of multiple microchannel potentials to produce the reference signal ensures the reference does not carry significant signal, so the source of the output signal from the amplifier is unambiguous.

In some embodiments, the recording array may be tripolar i.e. the recording array may comprise three electrodes connected by tripole amplifier circuit. A tripolar recording array may provide improved noise reduction.

The three electrodes may consist of a central electrode located symmetrically (i.e. equidistant) between two outer electrodes. In some preferred embodiments, the outer electrodes are spaced as far apart as possible, for example adjacent the ends of the microchannel. The central electrode may be referenced to connected (i.e. commoned) outer electrodes to produce a signal which is indicative of the potential in the microchannel between the central and outer electrodes.

A suitable tripole amplifier circuit may be selected from the group consisting of a quasi-tripole (QT) circuit, a true tripole (TT) circuit and an adaptive tripole (AT) circuit. QT, TT and AT amplifier circuits are well-known in the art (see for example; Triantis et al IEEE Trans Biomed Eng. 2005 February; 52(2):314-20).

An example of a tripolar recording array is shown in FIG. 47. The left side of the bridge circuit is formed by the impedances of ‘half-channels’ AC and CB and the right side of the bridge circuit is formed by impedances of the two end electrodes. Because external noise presents as a voltage between A and B; if R_{AC}=R_{CB} and the electrode impedances are matched, noise is cancelled as a common mode in the output signal from the amplifier.

The recording array may produce a signal which is indicative of the extracellular electrical signal in the microchannel. This signal may be subjected to processing and analysis as described below. A microchannel may comprise one or more, two or more, three or more or four or more recording arrays

The data shown herein also indicates that the signal generated in the extracellular fluid by action potentials from large diameter axons is greater than that generated by small diameter axons. Although motor axons generally have larger diameters than sensory axons, there are large and small motor axons which may have different functions. Furthermore sensory axons are of many sizes, the different sizes carrying different modalities of sensation; the smallest are pain fibres, the largest from muscle spindles. Characterising the amplitude of different action potentials may allow the functions and modalities of the axons in a particular microchannel to be identified.

Furthermore, in microchannels containing a variety of different axons, action potentials from a particular type of axon, for example a sensory or motor axon, may be selectively monitored by filtering the signals from the one or more electrodes, for example using threshold circuitry. Motor fibres give the largest amplitudes and may be selectively monitored by filtering out signals below a threshold amplitude. Sensory fibres give smaller amplitudes and may be selectively monitored by filtering out signals above a threshold amplitude. Sensory fibres specifically associated with tactile sensation give amplitudes that are intermediate between motor fibres and other sensory fibres, such as pain fibres, and may be selectively monitored by filtering out signals outside a particular amplitude range.

The actual signal amplitude which is generated by a nerve fibre in a microchannel is dependent on the diameter and length of the microchannel. The amplitude thresholds for different types of fibre in any particular microchannel may be readily determined using routine techniques as described herein.

The one or more electrodes may be connected to circuitry which filters out electrical signals detected in the microchannel which are above or below a threshold amplitude or outside an amplitude range, such that only electrical signals with amplitudes characteristic of a particular fibre type (e.g. motor nerve or sensory nerve) remain. After filtering, the signal may be indicative of action potentials in nerve fibres of a particular type, e.g. motor nerve or sensory nerve action potentials.

The one or more electrodes may be connected to additional circuitry for amplification, analogue-to-digital conversion and/or low-pass filtration to reduce noise and prevent aliasing.

One or more electrodes in a microchannel which generate an extracellular stimulus current suitable for inducing an action potential in an axon in the microchannel may be termed a stimulation array. A microchannel may comprise one or more, two or more, three or more or four or more stimulation arrays

The amount of stimulus current required to induce an action potential is shown herein to be reduced within the microchannel relative to outside the microchannel, due to the increased extracellular resistance within the microchannel. The amount of stimulus current required to induce an action potential is proportional to the cross-sectional area of the microchannel, so as the cross-sectional area of the microchannel small decreases, the amount of stimulus current required to induce an action potential also decreases.

The threshold stimulus current for inducing an action potential in an axon is also shown herein to be dependent on axon diameter. This allows selective stimulation of large diameter axons in the microchannel e.g. motor axons, for example by employing stimulus currents of a size sufficient to induce action potentials in large diameter axons but insufficient to induce action potentials in small diameter axons. Suitable stimulus currents may be readily determined for any particular microchannel using routine techniques as described herein.

In some embodiments, the action potential which is induced in the axon is bi-directional i.e. it propagates in both directions along the nerve axon and is both centrally- and peripherally-directed.

A stimulation array for bi-directional stimulation of action potentials may be monopolar, bipolar or tripolar.

A suitable monopolar stimulation array may comprise a single electrode such that the stimulus current passes between the electrode and an external ground

A suitable bipolar stimulation array may comprise a pair of electrodes axially spaced along the microchannel such that the stimulus current passes between the electrodes.

A suitable tripolar stimulation array may comprise a pair of anodes axially spaced along the microchannel and a cathode located between the anodes, such that the stimulus current passes between the cathode and the anodes. The cathode may be positioned symmetrically or asymmetrically between the anodes (i.e. located equidistant from the anodes or closer to one anode than the other).

In other embodiments, the action potential which is induced in the axon is uni-directional i.e. it propagates in one direction only along the nerve axon and is either centrally or peripherally directed.

Unidirectional stimulation allows modality-specific stimulation in microchannels containing nerve fibres of different types. For example, peripherally-directed action potentials activate muscles if they are in motor fibres, but have no effect in sensory axons; whereas centrally-directed action potentials are effective in sensory axons but have no effect in motor axons.

The F-wave is a weak delayed muscle contraction visible in the EMG which occurs when an antidromic action potential reaches large alpha motor cells in the spinal cord and reflects back towards the periphery. Unidirectional stimulation of motor axons may be useful in preventing the generation of an F-wave (as there is no centrally directed signal).

The stimulation of action potentials in a single direction in an axon also allows the collision blockade of undesirable action potentials in that axon. This is described in more detail below.

A unidirectional action potential may be induced in the axon by generating unequal stimulus currents in different parts of the microchannel. For example, a first stimulus current may be generated in a first region of the microchannel and a second stimulus current may be generated in a second region of the microchannel,
wherein the first and second regions are axially spaced in the microchannel and the first stimulus current is greater than the second stimulus current,
such that the action potential is induced in the axon in the direction from the first region to the second region of the microchannel.

Preferably, the first and second stimulus currents are maintained for at least 100 μs, at least 200 μs or at least 300 μs.

A suitable stimulus current for inducing a unidirectional action potential is above a threshold value and below an anode-break excitation value. Threshold values and anode-break excitation values will vary depending on the microchannel and electrode arrangement but may readily be determined for any given system using routine techniques.

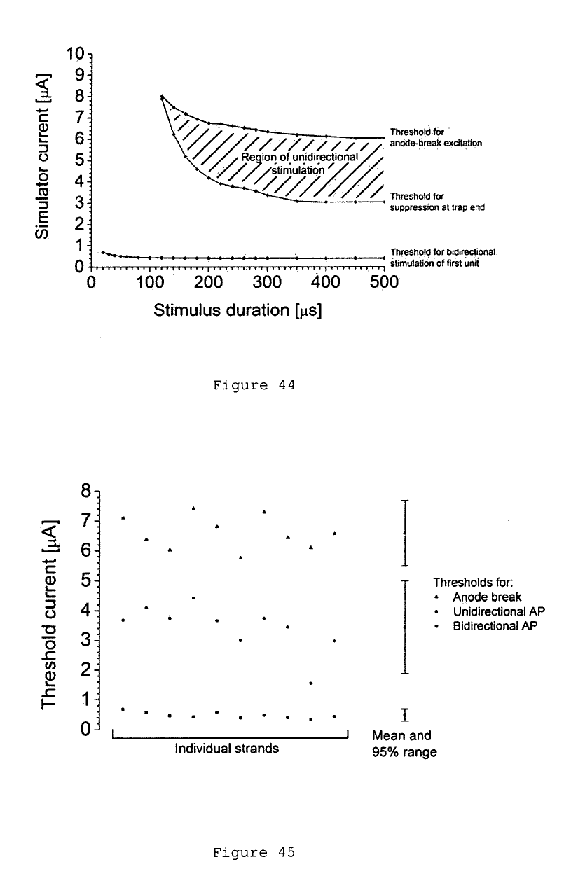

The amount and duration of the stimulus current may be optimised for any particular electrode and microchannel arrangement for unidirectional action potentials. For example, FIG. 44 shows the combinations of stimulus current amplitude and duration that provide unidirectional stimulation in the described electrode coupled microchannel. Similar graphs may be prepared to identify combinations of stimulus current amplitude and duration that provide unidirectional stimulation for other electrode coupled microchannel configurations.

A suitable stimulation array for uni-directional stimulation of action potentials may be monopolar or tripolar.

A monopolar stimulation array may comprise a cathode which is positioned asymmetrically between the ends of the microchannel (i.e. located closer to a proximal end than a distal end),
such that the first stimulus current between the cathode and the proximal end of the microchannel is greater than the second stimulus current between the cathode and the distal end of the microchannel, and;
the action potential is induced in the axon in the direction from the proximal end to the distal end of the microchannel.

In the monopolar configuration, the first and second stimulus currents flow between the single cathode and the anodes which are grounded outside the channel. The asymmetric position of the cathode generates stimulus currents of different sizes between the cathode and the proximal and distal ends of the microchannel.

A tripolar stimulation array may comprise first and second anodes axially spaced along the microchannel and a cathode positioned between the anodes,
such that the first stimulus current between the cathode and a first anode is greater than the second stimulus current between the cathode and the second anode and;
the action potential is induced in the axon in the direction from the first anode to the second anode of the microchannel.

In some embodiments, the first stimulus current and second stimulus currents may be generated by separate current sources which supply different amounts of current. For example, the cathode and the first anode may be connected to a first current source which provides the first stimulus current and the cathode and the second anode are connected to a second current source which provides the second stimulus current,
wherein the first current source provides a higher current than the second current source.

In other embodiments, the first stimulus current and second stimulus currents may be generated by the same current source. For example, the first and second anodes may be connected (i.e. commoned) and the cathode may be positioned asymmetrically between the anodes (i.e. located closer to the first (proximal) anode than the second (distal) anode), such that the first stimulus current between the cathode and the first anode is greater than the second stimulus current between the cathode and the second anode.

When the cathode is positioned asymmetrically between the anodes, the distance between the proximal anode and the cathode is less than half the distance between the proximal anode and the distal anode. For example, the distance between the proximal anode and the cathode may be less than 40%, less than 30%, less than 20% or less than 10% of the distance between the proximal anode and the distal anode.

Preferably, the distance between the proximal anode and the cathode is generally greater than the internode distance of the axon in the microchannel. For example, the distance between the proximal anode and the cathode may be greater than 300 μm.

The direction of the induced action potential in the axon depends on the relative arrangement of the axon and the stimulation array. For example, the stimulation array may be arranged in the microchannel such that the induced action potential is orthodromic (i.e. it propagates in the natural direction of action potentials in the axon; peripherally in a motor neuron or centrally in a sensory neuron) or antidromic (i.e. it propagates in the opposite direction to natural action potentials in the axon).

Antidromic action potentials induced in the axon may be used to block the passage of orthodromic action potentials in the axon. The action potentials propagate in opposite directions in the axon and are extinguished when they meet (collision blockade). This may be useful, for example, in blocking the passage of undesirable neural signals in an individual, such as pain signals or misrouted motor signals.

An antidromic action potential may be induced in the axon by the stimulation array in response to external input. For example, the production of antidromic action potentials may controlled by an operator (who may be the individual, a medical practitioner or another person). The stimulation array may produce a series of antidromic impulses in response to the external input and this series of impulses may be activated or deactivated by the operator through the external input. Alternatively, a processor or other device may activate the stimulation array to induce one or more antidromic impulses in accordance with a schedule or program.

An antidromic action potential may be induced in the axon by the stimulation array in response to an orthodromic action potential in the axon. For example, the electrode coupled microchannel may comprise a recording array and a stimulation array as described herein. The recording array is positioned in the microchannel to detect orthodromic action potentials in the axon before they reach the stimulation array (i.e. upstream side of the stimulation array with respect to an orthodromic signal) and the stimulation array is arranged to induce antidromic action potentials in the axon. The recording array detects an orthodromic action potential in the axon and triggers the induction of an antidromic action potential in the axon by the stimulation array in response. The induced antidromic action potential then blocks the passage of the orthodromic action potential through the interface.

Interfaces as described herein may be produced using any convenient technique. For example, a layer of dielectric substrate, such as polyimide, may be spin coated onto a rigid carrier, such as glass or silicon. A thin metal film may then be deposited onto the substrate using conventional sputter or evaporation techniques and the electrodes and leads produced from the film by patterning using photoresist, etching the metal film and removing the photoresist. Alternatively the electrodes and leads may be patterned by lift-off (i.e. first the photoresist is spin-coated and patterned, then the metal is deposited and the photoresist dissolved).

The electrodes and leads may then be encapsulated by a layer of insulating material by spin coating and the insulating material removed from the electrodes to expose them to the interior of the microchannel. The insulating material may also be removed from the contact pads of the output connector to allow for electrical connection of the interface to other devices.

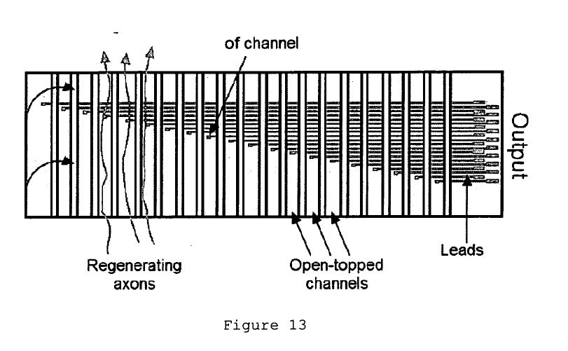

A microchannel layer of insulating material can then be spin-coated onto the encapsulated metal film and the microchannels patterned. A two dimensional sheet produced by such a method is shown schematically in FIG. 13 and an example is shown in FIG. 21.

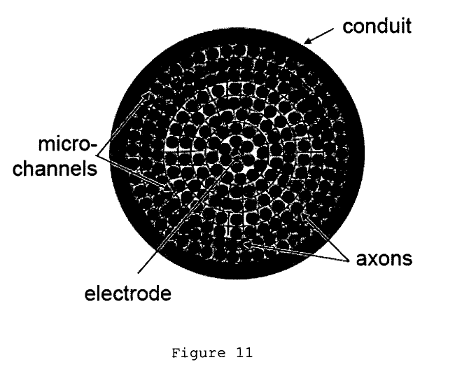

A three dimensional array of microchannels may be produced from a two dimensional sheet of electrode-coupled open grooves by any convenient technique. For example, the sheet may be rolled up so that the open grooves are closed by the next layer to form microchannels (see FIGS. 11, 14 and 26); the sheet of substrate may be covered with a sheet or strip of material, such as 4-fluorinated ethylene/6-fluorinated propylene copolymer (FEP), to close the grooves to form microchannels; or sheets of substrate may be stacked on top of each other, with each layer of open grooves being closed by the sheet above, to produce stacks of microchannels (FIG. 14).

Alternatively, the neural interface may be produced by casting a dielectric material, such as a silicone elastomer, with an array of threads passing through it. The threads are subsequently removed, leaving microchannels within the insulating material. This is shown in FIG. 16. The sieve transparency and geometry must exactly match the microchannel array (perfect alignment).

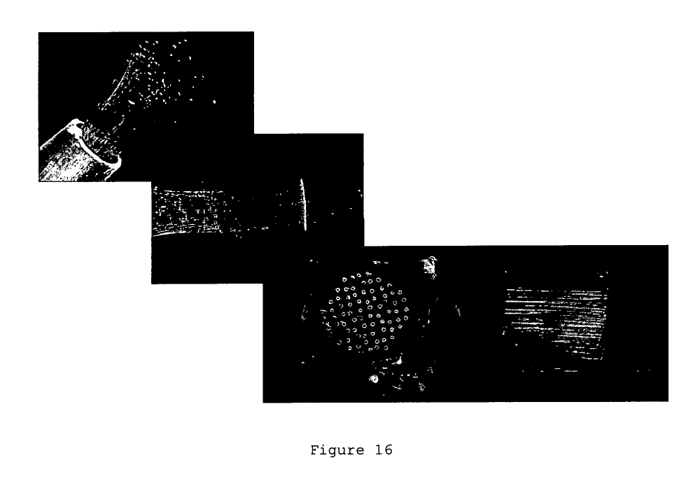

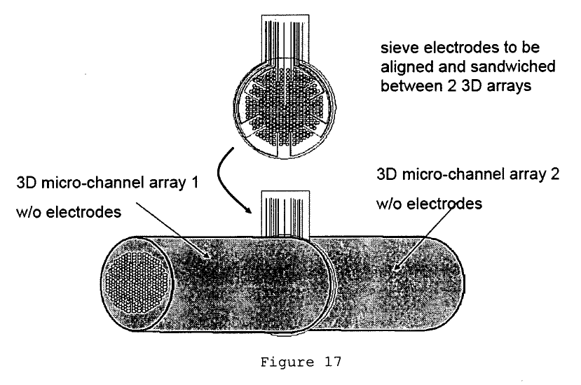

Electrodes may be added to the microchannels, for example, by introducing an array of sieve electrodes corresponding to the array of microchannels to the substrate, such that the microchannels in the array are associated with an electrode in the sieve array. Conveniently, this may be achieved by inserting the sieve array between two portions of the microchannel array (FIG. 17). Alternatively, the dielectric substrate may be cast with ring electrodes positioned around each thread in the array. When the threads are removed, the ring electrodes remain in position in the substrate, where they contact the resultant microchannel.

Alternatively, the neural interface may be produced by moulding from a silicon wafer. This involves etching a silicon wafer with a mask to the appropriate channel geometry; spin coating a polymeric material, such as polyimide, onto the silicon mould then curing and peeling off the polymer. To integrate the electrodes, an opening can be etched in the moulded sample, then the electrodes can be metallised and patterned on the back of the moulded sample and the electrodes then encapsulated in a dielectric material.

Alternatively, microchannels may be etched through a wafer of silicon (typically 250 um-1 mm thick) by dry (reactive ion etching RIE) or wet (anodisation) etching. To make channels longer than about 1 mm, several silicon wafers etched in this way may be fixed together.

All materials used to produce the electrode coupled microchannels are preferably resistant to sterilisation processes.

After production the interface may be sterilized using any convenient technique, such as autoclaving, irradiation or UV exposure.

Microchannels in the interfaces described herein preferably contain one or more biological factors which facilitate the regeneration of damaged nerve fibres from the proximal stump of a nerve, such as a peripheral nerve, to the distal stump.

Following sterilisation of the microchannels, biological factors may be incorporated using any convenient technique. For example, they may be coated onto the sides of the microchannels, for example using an adhesion molecule such as poly-D-lysine, and/or may be incorporated into a gel within the microchannels.

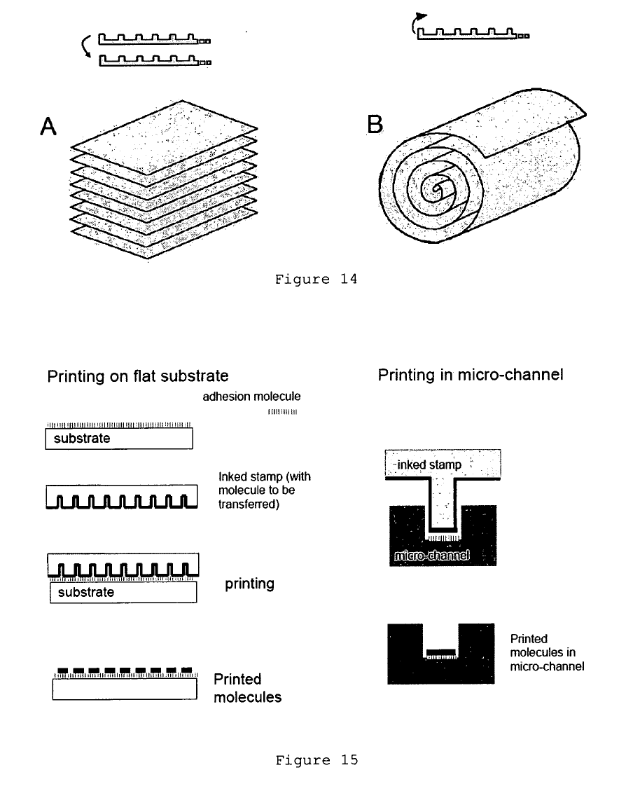

In some embodiments, microcontact printing (μCP) techniques may be employed. This involves activating the substrate with an adhesion molecule, such as poly-D-lysine. An elastomeric stamp is inked with the biological factor and printed onto the activated substrate. The biological factor then covalently attaches to the activated substrate. This allows precise patterns of factors to be fixed to the substrate. Substrates suitable for microcontact printing include flat substrates and microchannel substrates (FIG. 15).

Biological factors which may be employed include promoters of axon growth and Schwann cell invasion, such as laminin, fibronectin, collagen type 4, and heparan sulphate proteoglycans, or molecules comprising the axon growth promoting domains of those molecules.

The microchannels may contain a neurotrophic factor such as nerve growth factor, brain derived neurotrophic factor and/or neurotrophin 3.

To promote Schwann cell division and migration, Schwann cell growth factors such as neuregulins may be employed. For oligodendrocyte precursor migration and division, growth factors such as fibroblast growth factor 1 and platelet derived growth factor may be employed.

To promote the ingrowth of blood vessels, growth factors such as vascular endothelial growth factor (VEGF) may be employed.

Biological factors, such as neutralising antibodies to TGFbeta, decorin and corticosteroids, may be employed to limit inflammation and scarring.

Specific axon guidance molecules, such as semaphorin 3A, netrins, slits (e.g. Slit-1), ephs, ephrins and other forms of semaphorin may be employed to select for specific modalities of axon For example, semaphorin 3A repels pain axons but not axons from muscle spindles and so may be used to specifically guide muscle spindle axons into a microchannel.

The surfaces of the microchannels may be modified to promote the regeneration of axons and glial cells and to increase the biological compatibility of the interface. For example, the internal surface of the microchannel may be textured or modified to provide optimal conditions for axon regeneration. In some embodiments, the roughness of the surface may be increased to promote axon growth and cell migration. The surface of a polymeric insulating material may be roughened by plasma treatment such as oxygen plasma, or by nano-imprinting or embossing. Embossing involves pressing a master with a nanoscale pattern onto the surface of a polymeric material (usually slightly heated to increase its viscosity) and removing the master to leaving an imprinted copy of the master on the surface of the polymeric material.

The hardness of the surface may also be modified to improve the growth and migration of glial cells and axons. The elastic storage modulus of cells is between 100 and 3000, so the surface properties of the microchannels may typically be varied within this range to match that of the cells that adhere to them. Surfaces of different softness may be produced, for example in polyacrylamide gels or other forms of gel which can be coated onto the inner surface of the microchannel.

An interface as described herein is preferably contained within an outer tube. This allows the damaged ends of the nerves to be inserted and preferably attached to each end of the tube, such that the regenerating axons can enter the microchannels of the interface.

The proximal end of the damaged nerve may, for example, be attached to the outer tube with sutures or glue or biological glues such as thrombin.

The diameter of the outer tube is preferably matched to the nerve to which it is being attached and will vary from nerve to nerve. Typically, a diameter of 2-10 mm may be useful in many applications.

The outer tube may, for example, be made of a biocompatible plastic such as silicone tubing, a permeable or microporous biocompatible material such as polylactone, a resorbable material, or from a biological material such as collagen tubing (which is already sold commercially for nerve repair).

In some embodiments, the interface may be contained in a conductive shell. The conductive shell may be positioned outside the outer tube and may be useful in shielding the interface from electromagnetic radiation, thereby reducing noise. The conductive shell may be made of any conductive material, for example a metal such as stainless steel or titanium.

Following attachment to a damaged nerve, the interface is left in situ to allow the regenerating axons to enter the microchannels. Action potentials may be detected once axons have entered the microchannels. This may take several weeks.

The electrodes are connected via leads, which are fabricated into the device, to the pads of an output connector. The output connector may, in turn, be connected to off-board electronic devices (for example, wirelessly or via a flexible or stretchable cable) which receive and process the electrical information coming from the nerve implant, and convert it into a form that can be transmitted for further processing, by processors or actuators outside or inside the body.

The output connectors may be connected to one or more implantable electronic devices which may receive and process the electrical information from the interface and transmit it to off-board electronics for further processing and/or to a user interface.

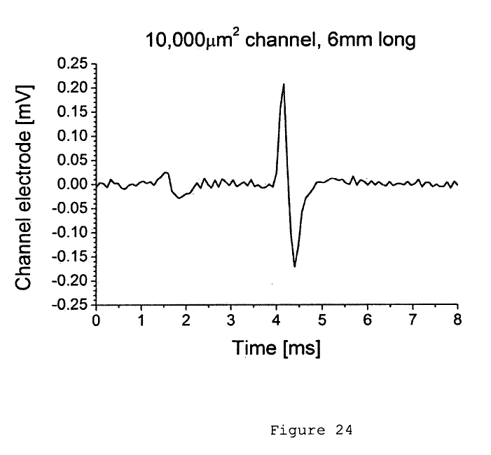

The output connectors may be connected to a wireless (telemetric) transmitter which transmits the information for further processing. Examples of wireless and non-wireless embodiments are shown in FIGS. 23 and 24.

The electrodes may be operably linked to a processor through the output connector.

The processor may be operably linked to a recording array in the microchannel. The processor may be programmed to record, process and/or analyse the signals from the recording array. For example, the processor may filter, amplify, and/or digitise the signals from the recording array. The processor may be programmed to characterise the action potentials in the axon from said signals. The axial spacing of electrodes in the microchannels described herein allows the direction and velocity of the action potentials in the microchannel to be determined, as well as their frequency and pattern. Furthermore, the electrode-coupled microchannels described herein display a reliable relationship between axon size and signal amplitude. Measurement of signal amplitude using the electrode-coupled microchannels therefore allows different axons in the same microchannel to be differentiated and the size and likely modality of each axon determined.

The processor may be operably linked to a stimulation array in the microchannel. The processor may be programmed to activate the stimulation array to induce action potentials in the axons in the microchannel. The processor may be programmed to stimulate action potentials in the axon with specific characteristics. For example, the processor may determine one or more of the amplitude, direction, speed, frequency and pattern of the action potentials which are induced in the microchannel. In some embodiments, the processor may be programmed to stimulate action potentials in the axon in accordance with a programme or schedule. The programme or schedule may be programmed into the processor by the operator. In some embodiments, the processor may be programmed to stimulate action potentials in an axon in response to action potentials detected in the axon by a recording array which is operably linked to the processor.

The information from the one or more electrodes may be used, for example, to drive limb prostheses in response to the neural information from the interface, or to drive implanted electrodes in muscles (to stimulate muscle contraction when appropriate axons in the prosthesis are active). The information may also be used to drive stimulators of sensory axons in the peripheral nervous system around sensory nerves, or devices implanted in or adjacent to the spinal cord or sensory regions of the brain.

In some embodiments, the processor may be programmed to translate the patterns of action potentials recorded in the microchannels into signals which instruct the action of particular muscle stimulators or prosthetic actuators. This may, for example, be performed using an electronic neural network. In some embodiments, following implantation of the interface and regeneration of the axons through it, there may be an initial learning phase where software is ‘trained’ to recognise specific patterns of action potentials recorded in the microchannels as being instructions for certain movements of the limb or prosthetic. For example, the patient may perform a defined movement and the pattern of action potentials recorded in the microchannels as a result of the defined movement is recorded and stored for future recognition. Likewise for sensory feedback: for each desired sensation, trials would be done with the patient during the initial learning phase to identify which microchannels must be stimulated in the interface to elicit particular sensations for the patient.

Other aspects of the invention relate to the detection and/or measurement of action potentials in nerve axons using microchannels as described herein.

A method of detecting and/or measuring an action potential in a nerve axon may comprise;
providing a microchannel containing a nerve axon in an conductive medium, and
detecting changes in electrical signal in the medium, said changes being indicative of an action potential in the nerve axon.

The changes in electrical signal in the medium may be detected using one or more electrodes which are in electrical contact with the medium within the microchannel. As described above, the electrical signals are independent of the positions of the nodes of the nerve axon in the microchannel relative to the electrodes.

Suitable microchannel and electrodes are described above. The microchannel may be comprised in a neural interface as described above. The microchannel may be used in vitro or in vivo in accordance with the present methods.

As described above, the signal detected by the electrodes may be amplified and/or filtered according to predetermined amplitude criteria. For example, signals below a threshold amplitude may be filtered out to selectively detect motor fibre signals; signals above a threshold amplitude may be filtered out to selectively detect sensory fibre signals and signals outside a particular amplitude range may be filtered out to selectively detect signals from particular types of sensory fibre, such as sensory fibres specifically associated with tactile sensation. The signal may be further processed as required, for example using amplification, digitisation or noise reduction techniques.

The signals may be recorded in a processor. Filtration, amplification, digitisation and other processing of the signals from the recording array may also be performed in the processor.

The signals may be analysed to characterise the action potentials in the axon. For example, one or more of the amplitude, direction, speed, frequency and pattern of the action potentials in the microchannel may be determined from the signals. The function or modality of the axon in the microchannel may be identified from the characterised action potentials. Preferably, analysis and characterisation is performed in a processor.

Other aspects of the invention relate to the stimulation of changes in membrane potential in neuronal processes using microchannels as described herein.

A method of stimulating a change in membrane potential in a neuronal process may comprise;
providing a microchannel containing a neuronal process in a conductive medium, and
one or more electrodes exposed to the interior of said microchannel
wherein the one or more electrodes form a stimulation array which produces an extracellular stimulus current in the conductive medium which induces a change in membrane potential in the neuronal process.

For example, the above methods may be useful in stimulating an action potential in a nerve axon.

Suitable microchannels, electrodes and neural interfaces are described above. The microchannel may be used in vitro or in vivo in accordance with the present methods

The action potential induced in the axon may be bi-directional or uni-directional. The generation of bi-directional or uni-directional action potentials is described in more detail above.

Other aspects of the invention relate to the collision blockade of orthodromic action potentials nerve axons.

A method of blocking an orthodromic action potential in a nerve axon may comprise;
providing a microchannel containing a nerve axon in a conductive medium, and having a stimulation array comprising one or more electrodes contacting the conductive medium,
said stimulation array being arranged so as to induce antidromic action potentials in the nerve axon, and
   generating a stimulus current in the microchannel through stimulation array to induce an antidromic action potential in the nerve axon,
wherein the antidromic action potential induced in the axon blocks the passage of an orthodromic action potential in the axon.

Suitable stimulus currents to evoke antidromic action potentials in any particular electrode-coupled microchannel arrangement may be readily determined using routine techniques as described herein.

In some embodiments, orthodromic action potentials may be detected in the axon by a recording array. For example, the electrode coupled microchannel may include a recording array comprising one or more electrodes which contact the conductive medium and the method of method of blocking an orthodromic action potential may comprise detecting the orthodromic action potential in the axon in the microchannel using the recording array.

Detection of the orthodromic action potential by the recording array then triggers the stimulation array to induce the antidromic action potential.

The recording and stimulation arrays may be operably linked to a processor which controls the detection of orthodromic action potentials and the stimulation of antidromic action potentials.

Other aspects of the invention relate to methods of detecting, measuring and/or stimulating an action potential in a regenerated nerve axon.

A method of detecting and/or measuring an action potential in a nerve axon may comprise;
positioning a neural interface as described above at the proximal stump of a damaged nerve,
allowing an axon of said damaged nerve to regenerate from the proximal stump into a microchannel of the interface in the presence of a conductive medium, and
detecting changes in electrical signal in the medium within the microchannel, said changes being indicative of an action potential propagating along the nerve axon.

A method of detecting and/or measuring an action potential in a nerve axon may comprise;
providing a neural interface as described above which is positioned at the proximal stump of a damaged nerve,
wherein a microchannel of said interface accommodates, in a conductive medium, an axon of said damaged nerve which has regenerated from the proximal stump, and
detecting electrical signals in the medium within the microchannel, said changes being indicative of the propagation of an action potential in the regenerated nerve axon.

A method of stimulating an action potential in a nerve axon may comprise
positioning a neural interface as described above at the proximal stump of a damaged nerve,
allowing an axon of said damaged nerve to regenerate from the proximal stump into a microchannel of the interface in the presence of a conductive medium, and
generating an extracellular stimulus current in the conductive medium which induces an action potential in the nerve axon.

A method of stimulating an action potential in a nerve axon may comprise;
providing a damaged nerve having a neural interface as described above positioned at the proximal stump thereof,
wherein a microchannel of said interface accommodates, in a conductive medium, an axon of said damaged nerve which has regenerated from the proximal stump, and
generating an extracellular stimulus current in the conductive medium which induces an action potential in the nerve axon.

Suitable neural interfaces and the detection and stimulation of action potentials are described in more detail above.

The neural interface may be attached to the proximal stump and, optionally the distal stump, of the damaged nerve using sutures or adhesives as described above.

Axon of a damaged nerve may take several weeks to regenerate from the proximal stump into the microchannels of the interface.

Once axons have regenerated through the microchannels, the interface may be connected to additional electronic components such as a processor via the output connector. For example, connectors may be plugged into the array and connected to a processor or a wireless transmitter may be plugged into the array for wireless connection to the processor.

The interface may be kept in position indefinitely to detect and/or allow for stimulation of action potentials in regenerated nerve axons

As described above, typically, the electrical signal detected by the one or more electrodes in the medium may be subjected to low-pass filtering to reduce noise and prevent aliasing, followed by analogue-to-digital conversion.

The electrical signal may be processed to identify the size, direction, speed, frequency and pattern of the action potentials in the microchannel. This may be carried out by a processor integrated within the interface or connected to it as described herein.

This characterisation of the action potentials may enable the function and modality of the axon(s) in the microchannel to be identified. For example, the axon may be identified as a sensory or motor axon.

The detection of action potentials in motor axons may be used to drive prostheses. For example, action potentials detected in motor axons may be coupled to the control of a limb prosthetic. A method may comprise recording the pattern of action potentials in the microchannels of a neural interface as described herein, filtering the action potentials to obtain the pattern of motor fibre action potentials only and translating the pattern of motor fibre action potentials into instructions for contraction of various muscles or activation of prosthetic actuators. The pattern of motor fibre action potentials may, for example, be translated using a processor programmed with suitable pattern recognition software. Suitable pattern recognition software may include a neural network which is trained to recognise and translate pattern of motor fibre action potentials recorded in the interface.

A method of controlling a prosthesis may comprise;
providing a neural interface as described herein which is implanted into the individual having the prosthesis, wherein the microchannels of the interface accommodate motor nerve axons of the individual suitable for controlling the prosthesis,
detecting electrical signals in the medium within the microchannel which are indicative of the propagation of an action potential in the motor nerve axons, and
coupling the electrical signals detected in the medium with one or more actuators of the prosthesis,
such that action potentials in the motor nerve axons control the actuator mediated movement of the prosthesis.

The one or more actuators of the prosthesis may be coupled to the electrical signals detected in the medium through a processor which translates patterns of action potentials detected in the motor nerve axons into instructions for stimulation of the appropriate actuators of the prosthesis, as described above. The interface may be connected to the prosthesis by any convenient method, including physical connection, for example using wires, or wireless connection using a telemetric transmitter and receiver, as described herein.

Motor nerve axons of the individual suitable for controlling the prosthesis may include the motor nerve axons which controlled the limb or body part replaced by the prosthesis.

A prosthesis may include a prosthetic limb such as a prosthetic arm, leg hand or foot.

The detection of action potentials in motor axons may be used to improve the control of an individual's muscles after nerve repair. For example, action potentials detected in motor axons may be coupled to one or more muscle stimulators in a muscle targeting by the motor axon. This may be useful, for example, in the treatment of nerve injury.

A method of treatment of nerve injury may comprise;
positioning a neural interface as described herein at the proximal stump of a damaged nerve,
allowing one or more axons of said damaged nerve to regenerate from the proximal stump into a microchannel of the neural interface in the presence of a conductive medium,
detecting electrical signals in the medium within the microchannel which are indicative of the propagation of an action potential in the motor nerve axons, and
coupling the electrical signals detected in the medium with a muscle stimulator in a muscle targeting by the motor axon,
such that action potentials in the motor nerve axons stimulate the muscle targeting by the motor axon.

A muscle stimulator is a device for electrically stimulating muscle. Suitable stimulators are well known in the art and may comprise stimulating electrode(s) in contact with or buried into the muscle, a power source, and circuitry to create appropriate stimulus pulses in the electrode(s) using power from the power source.

The muscle stimulator may be coupled to the electrical signals detected in the medium through a processor which translates patterns of action potentials detected in the motor nerve axons into instructions for stimulation of the muscle targeting by the motor axon, as described above.

The detection of action potentials in sensory axons may be used to provide sensory input to the nervous system, for sensation and/or proprioception from a prosthesis

Sensors in a prosthesis may be coupled to the stimulation of electrical signals in the medium which induce action potentials in sensory axons.

A method of providing sensation and/or proprioception from a prosthesis may comprise;
providing a neural interface as described herein which is implanted into an individual having a prosthesis, wherein the microchannels of the interface accommodate comprise sensory nerve axons suitable for transmission of sensation and/or proprioception from the prosthesis,
coupling the sensors of the prosthesis to the interface such that signals from the sensors cause the stimulation array of the interface to generate a stimulus current in the medium within the microchannel which induces the propagation of an action potential in the sensory nerve axons,
said action potential providing sensation and/or proprioception to the individual from the prosthesis.

The sensors in the prosthesis may be coupled to the interface through a processor which translates signals and/or information from the sensors in the prosthesis into instructions for the stimulation of a pattern of action potentials in the sensory nerve axons. The interface may be connected to the prosthesis by any convenient method, including physical connection, for example using wires, or wireless connection using a telemetric transmitter and receiver, as described herein.

Sensory nerve axons suitable for transmission of sensation and/or proprioception from the prosthesis may include sensory nerve axons which transmitted sensation and/or proprioception from the limb or body part which was replaced by the prosthesis.

The stimulation of action potentials in sensory axons may be used to provide sensation and/or proprioception from denervated tissue following nerve injury or damage.

A method of repair of denervated tissue may comprise;
positioning a neural interface as described herein at the proximal stump of a damaged nerve,
allowing one or more axons of said damaged nerve to regenerate from the proximal stump into a microchannel of the neural interface in the presence of a conductive medium,
coupling sensors in the denervated tissue in the individual to the interface such that signals from the sensors stimulate electrical signals in the medium within the microchannel which induce the propagation of an action potential in the sensory nerve axons,
said action potentials providing feedback to the individual of sensation and/or proprioception from the denervated tissue.

The stimulation of antidromic action potentials in motor axons may be used to block signals in motor nerves which have reconnected to the wrong target muscle following mis-repair of nerve injury or damage.

A method of treating misrouted motor nerves may comprise;
positioning a neural interface described herein at the proximal stump of a damaged nerve,
allowing one or more proximal axons of said damaged nerve to regenerate from the proximal stump through a microchannel of the neural interface in the presence of a conductive medium into the distal stump of a damage nerve, wherein one or more of said regenerated proximal axons is misrouted to the wrong distal nerve fibre,
detecting electrical signals in the medium within the microchannel which are indicative of the propagation of an orthodromic action potential in the misrouted motor nerve axon, and
stimulating the interface to produce an antidromic action potential which blocks the orthodromic action potential in the misrouted motor nerve axon.

On detecting the electrical signal in said microchannel, the production of an orthodromic action potential may be stimulated in a second motor neuron axon accommodated in a different microchannel. This second motor neuron axon may be the connected to appropriate distal nerve fibre to stimulate the target muscle.

The detection and stimulation of action potentials may be controlled by a processor, as described above. For example, action potentials detected in the first microchannel may cause the processor to simultaneously trigger unidirectional stimulation for blockade in the first microchannel and stimulation in the second microchannel.

The stimulation of antidromic action potentials in sensory axons may also be used to provide block pain signals, for example from neuromas.

A method of blocking neuromal pain may comprise;
positioning a neural interface described herein at the proximal stump of a damaged nerve,
allowing one or more sensory nerve axons of said damaged nerve to regenerate from the proximal stump into a microchannel of the neural interface in the presence of a conductive medium such that a neuroma forms on the distal side of the interface,
detecting electrical signals in the medium within the microchannel which are indicative of the propagation of an orthodromic action potential in the sensory nerve axons from the neuroma, and
stimulating the interface to produce an antidromic action potential towards the distal side of the interface which extinguishes the orthodromic action potential in the sensory nerve axons from the neuroma.

A method of blocking pain may comprise;
positioning a neural interface described herein at the proximal stump of a damaged nerve,
allowing one or more sensory nerve axons of said damaged nerve to regenerate from the proximal stump into a microchannel of the neural interface in the presence of a conductive medium such that a pain source is located on the distal side of the interface, and
stimulating the interface to produce an action potential towards the distal side of the interface which extinguishes action potentials in the sensory nerve axons from the pain source.

The pain source may be a neuroma.

The interface may be stimulated to produce a continuous series of action potential towards the distal side of the interface which extinguish any action potentials in the sensory nerve axons from the neuroma.

Other aspects of the invention relate to neural interfaces as described for use in the above methods.

Other aspects of the invention relate to the use of electrode-coupled microchannels as described herein to study electrical interactions between cells by recording from or stimulating cell processes (axons or dendrites) in vitro. This may be useful in the study of electrical communication between neurons and neural computation.

An in vitro culture vessel may comprise;
a nerve cell depot linked to one or more electrode-coupled microchannels,
each electrode-coupled microchannel comprising;
a microchannel for accommodating neuronal processes extending from nerve cells in the cell depot; and
one or more electrodes exposed to the interior of said microchannel, such that electrical signals in the microchannel may be detected or stimulated in the processes by the electrodes.

The vessel may comprise multiple nerve cell depots all linked to one or more electrode-coupled microchannels.

In some embodiments, an electrode-coupled microchannel may link two or more nerve cell depots, such that neurons in the depots may extend processes to contact each other.

A nerve cell depot may be a well, recess, tube, dish or other containment which is capable of retaining nerve cells in a suitable growth medium. For example, a culture vessel may comprise an array of wells into which neurons are placed, the wells being linked by microchannels, along which the neurons could extend processes to contact each other.

The methods described herein may be useful in detecting, measuring and/or stimulating changes in membrane potential in a neuronal process in vitro.

A method of detecting and/or measuring action potentials in a nerve axon in vitro may comprise;
positioning an neural interface as described herein adjacent a nerve cell depot in vitro,
allowing an axon from a nerve cell in said depot to grow from the depot into a microchannel of the neural interface in the presence of a conductive medium, and
detecting electrical signals in the medium within the microchannel indicative of action potentials in the nerve axon.

In some embodiments, the changes in membrane potential may be action potentials and the neuronal process may be a nerve axon.

A method of stimulating a neuronal process in vitro may comprise;
positioning an neural interface as described herein adjacent a nerve cell depot in vitro,
allowing a neuronal process from a nerve cell in said depot to grow from the depot into a microchannel of the neural interface in the presence of a conductive medium, and
stimulating an electrical signal in the medium within the microchannel through the one or more electrodes which induces a change in membrane potential in the neuronal process.

In some embodiments, the changes in membrane potential may be action potentials and the neuronal process may be a nerve axon.

Various further aspects and embodiments of the present invention will be apparent to those skilled in the art in view of the present disclosure. All documents mentioned in this specification are incorporated herein by reference in their entirety.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Certain aspects and embodiments of the invention will now be illustrated by way of example and with reference to the figures described above and tables described below.

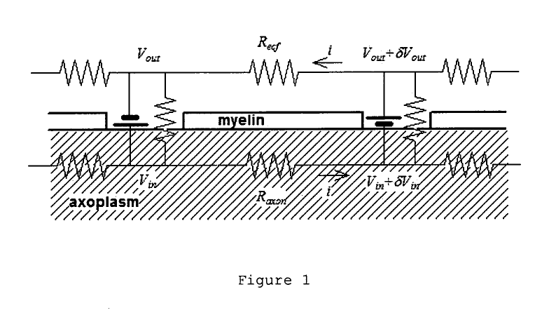

FIG. 1 shows local currents during depolarisation of an axonal membrane.

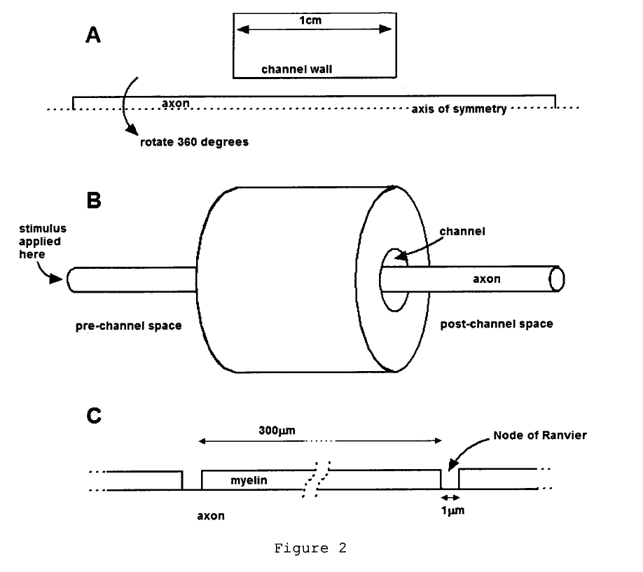

FIG. 2 shows simulation geometry. FIG. 2A shows a 2-dimensional axisymmetric geometry (unmyelinated model). Calculations are performed by the software as if the shapes drawn were swept 360° about the axis of symmetry to form the 3-dimensional structure depicted in FIG. 2B. FIG. 2C shows myelin segments added to axonal membrane for the myelinated model.

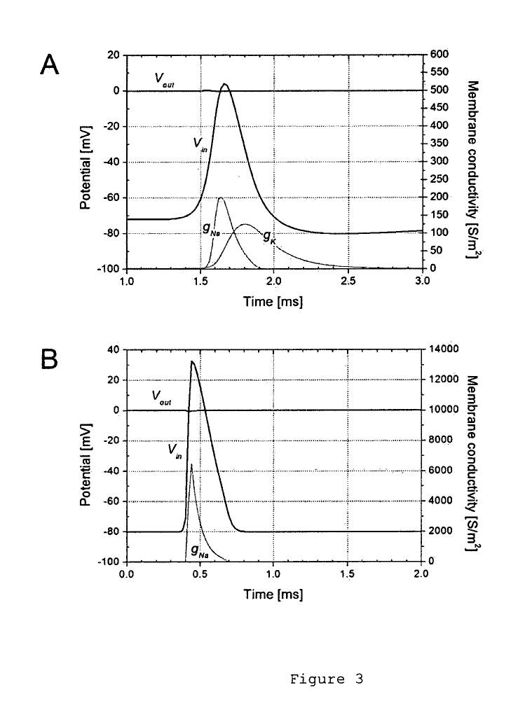

FIG. 3 shows action potential waveforms and underlying ionic conductances for a 2 μm unmyelinated axon (Hodgkin-Huxley model [14]: FIG. 2A) and a 10 μm myelinated axon (Chiu-Sweeney model [19]: FIG. 2B). In both cases, axons are surrounded by “infinite” extra-cellular fluid.

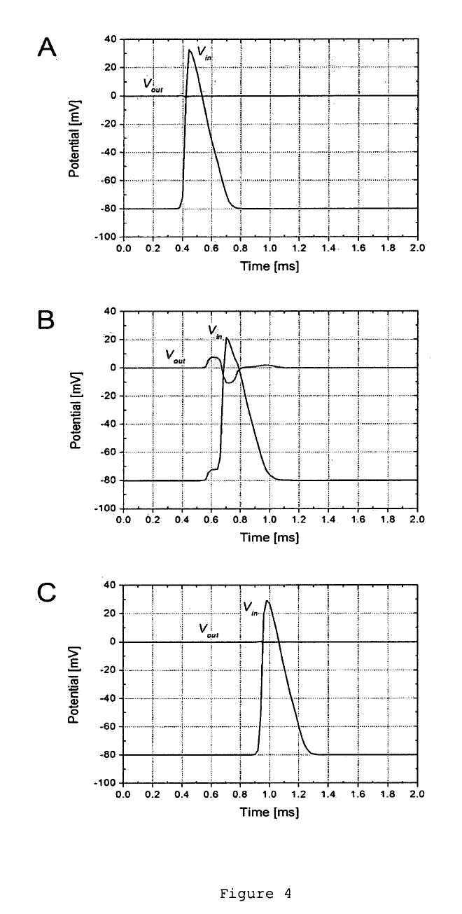

FIG. 4 shows intracellular (Vᵢₙ) and extracellular (Vₒᵤₜ) voltages for a 10 μm myelinated axon (FIG. 4A) 1 cm before, (FIG. 4B) midway along, and (FIG. 4C) 1 cm after passage through a 1000 μm² channel.

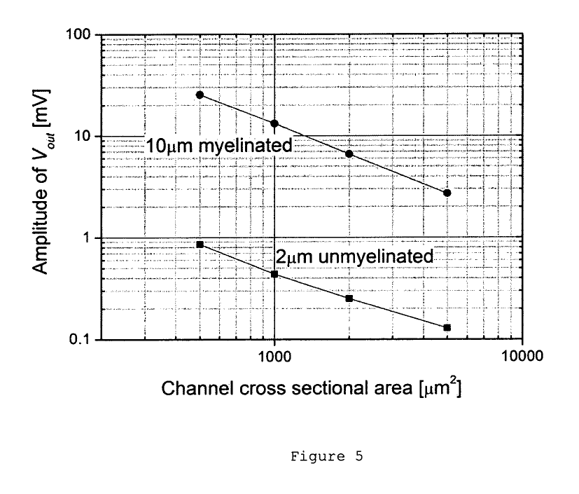

FIG. 5 shows amplitude of Vₒᵤₜ versus channel cross section.

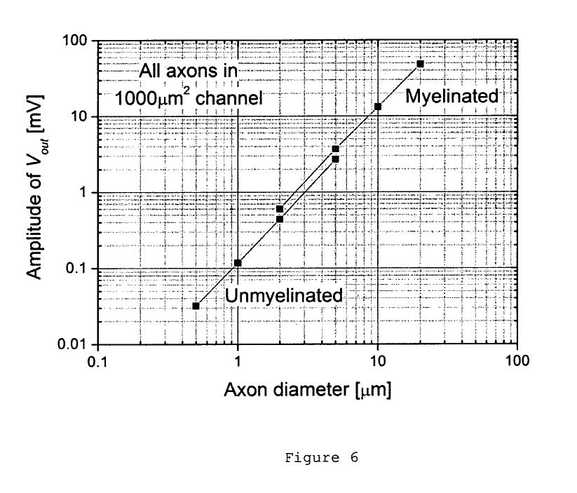

FIG. 6 shows amplitude of Vₒᵤₜ versus axon diameter.

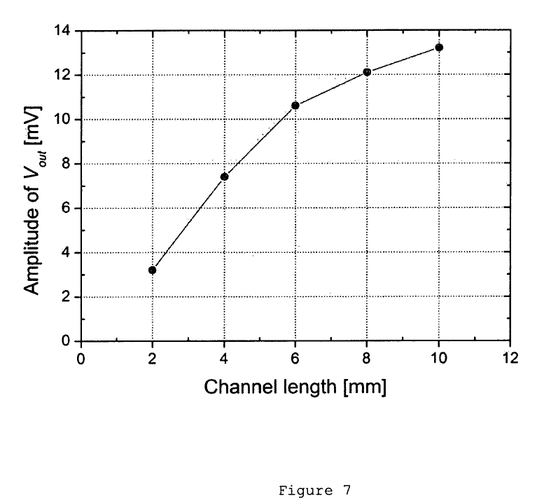

FIG. 7 shows amplitude of Vₒᵤₜ versus channel length (10 μm diameter myelinated axon in a 1000 μm2 channel).

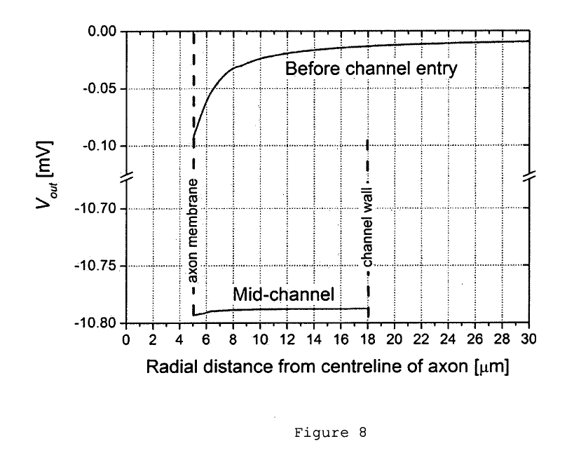

FIG. 8 shows amplitude of Vₒᵤₜ versus radial distance from centreline of a 10 μm myelinated axon in a 1000 μm² channel. Upper trace shows unconfined axon prior to channel entry, showing small signal and rapid decay with radial distance. Lower trace shows the same axon at mid-channel showing amplified signal (note the break in the vertical axis) with much less radial decay. For comparison, in both cases the measurements are opposite a node of Ranvier and are taken at the time of the signal peak at each point.

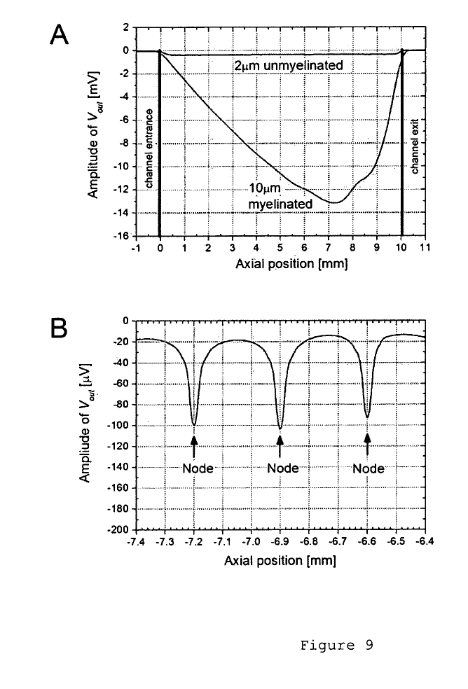

FIG. 9 shows amplitude of Vₒᵤₜ versus axial position in a 1000 μm² channel. FIG. 9A shows a 2 μm unmyelinated axon, showing a plateau in amplitude over most of the length of the channel, and a 10 μm myelinated axon, showing a maximum amplitude about 7 mm into the channel. FIG. 9B shows an amplitude close to a 1 mm section (note the different horizontal and vertical scales) of the same 10 μm myelinated axon approximately 7 mm before channel entry, showing the strong dependence of Vₒᵤₜ on node position; compare FIG. 9A where there is no evidence of dependence on node position within the channel.

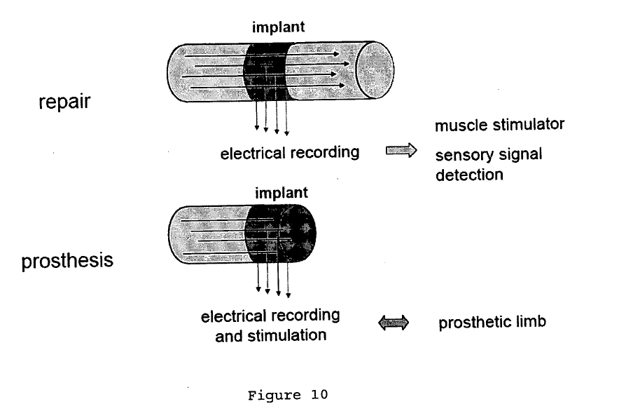

FIG. 10 shows different configurations of interfaces of the invention.

FIG. 11 shows a cross section of a 3D implant rolled interface held in a conduit. Note that only a few electrodes are represented and blood vessels are not shown.

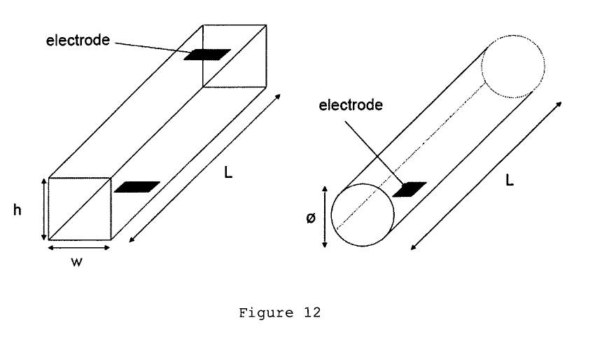

FIG. 12 shows the examples of electrodes positioned in a microchannel

FIG. 13 shows a substrate sheet with microchannels and electrodes prior to rolling into a 3D device.

FIG. 14 shows the fabrication of implants by stacking or rolling 2D sheets.

FIG. 15 shows a schematic of methods of printing biological factors onto the substrate.

FIG. 16 shows the fabrication of 3D microchannel arrays by passing fibres through two fine grids, separating the grids, pouring and curing the PDMS substrate, and withdrawing the fibres and cutting the block to size.

FIG. 17 shows techniques for the incorporation of electrodes into a 3D array of microchannels.

FIG. 18 shows action potentials in microchannels. FIG. 18A shows an action potential simulated from a 2 μm myelinated axon in 8,400 μm², 9 mm long channel. FIG. 18B shows an experimentally recorded action potential recorded from a myelinated axon in 8,400 μm², 9 mm long channel.

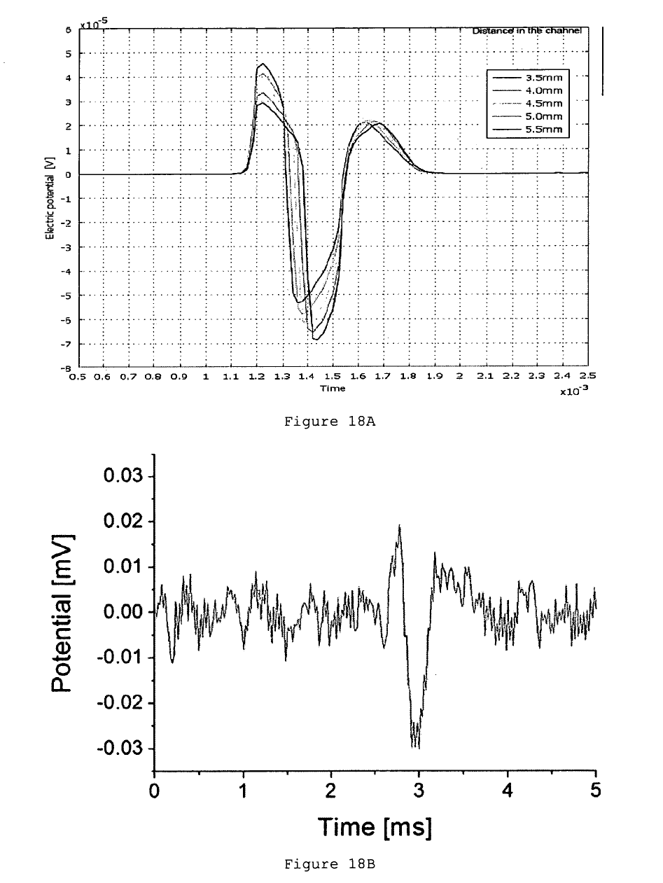

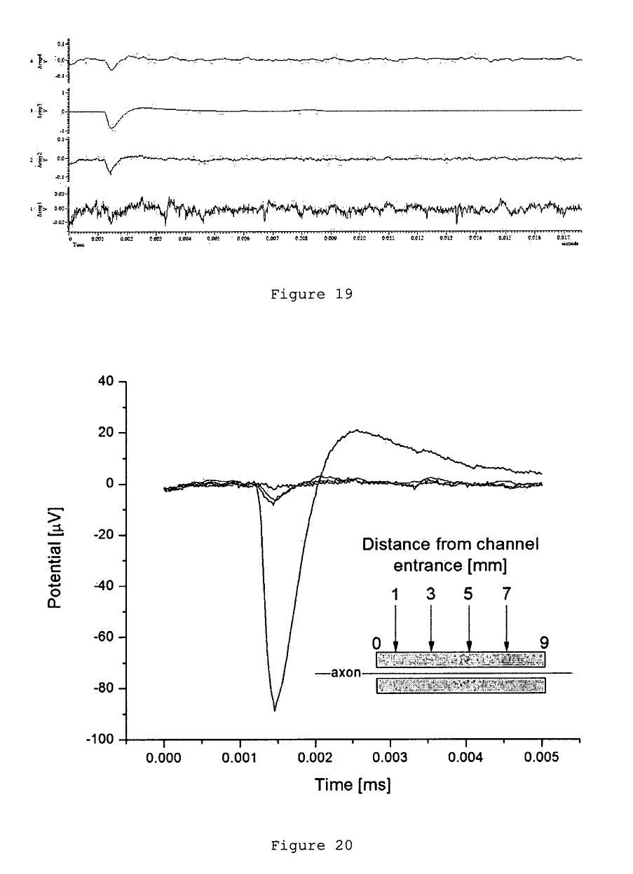

FIGS. 19 and 20 show action potentials recorded by 4 electrodes in a single channel.

FIG. 21 shows axons growing in microchannels.

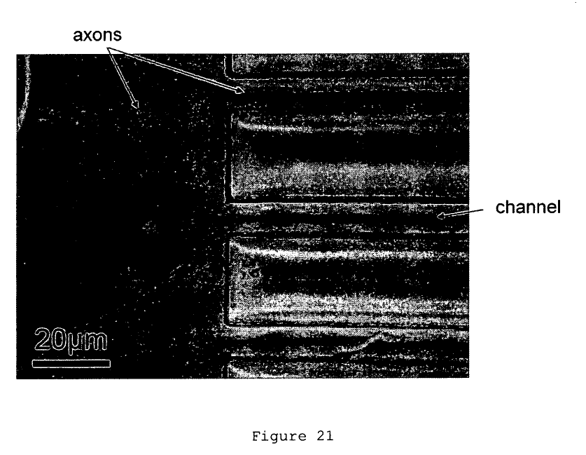

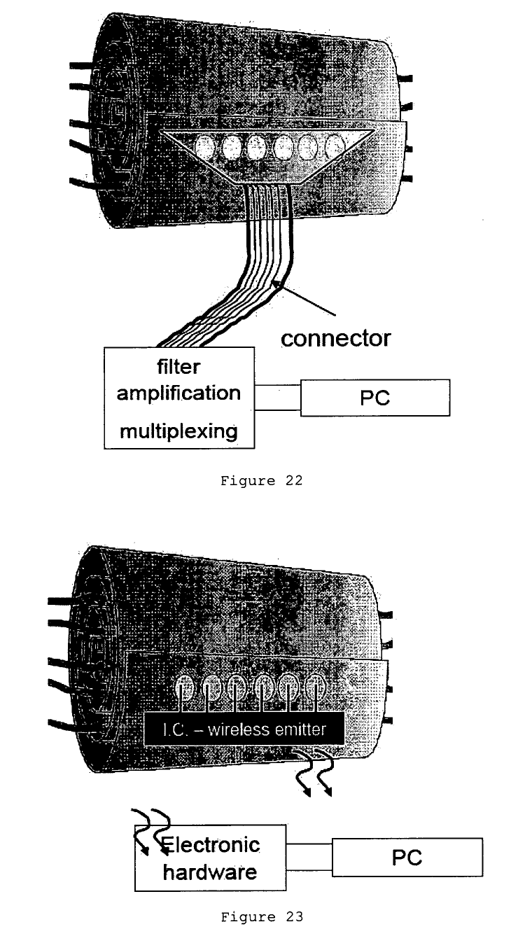

FIG. 22 shows an implant connected to electronic hardware via a connector.

FIG. 23 shows an implant connected to electronic hardware via a wireless link.

FIG. 24 shows an action potential with voltage >10 s μV recorded from a nerve strand enclosed in a micro-channel.

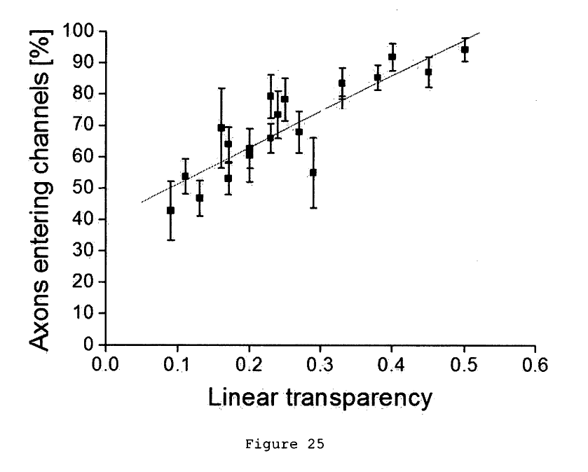

FIG. 25 shows the relationship between the linear transparency of an interface and the % of axons entering microchannels.

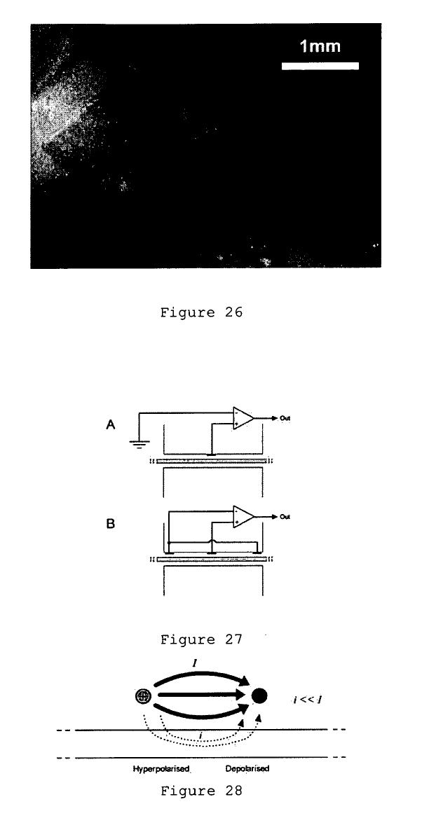

FIG. 26 shows a rolled interface device in a silicone tubing outer layer.

FIG. 27 shows electrode configurations for microchannel recording. A: Recording with a single in-channel electrode versus ground. B: Tripolar differential recording for noise reduction.

FIG. 28 shows large stimulus current (I) generates a potential gradient in the extracellular space that drives a much smaller transmembrane current (i). The membrane is depolarised near the cathode and hyperpolarised near the anode.

FIG. 29 shows electrode configurations for microchannel stimulation. A: Unipolar; B: Bipolar; C: Tripolar.

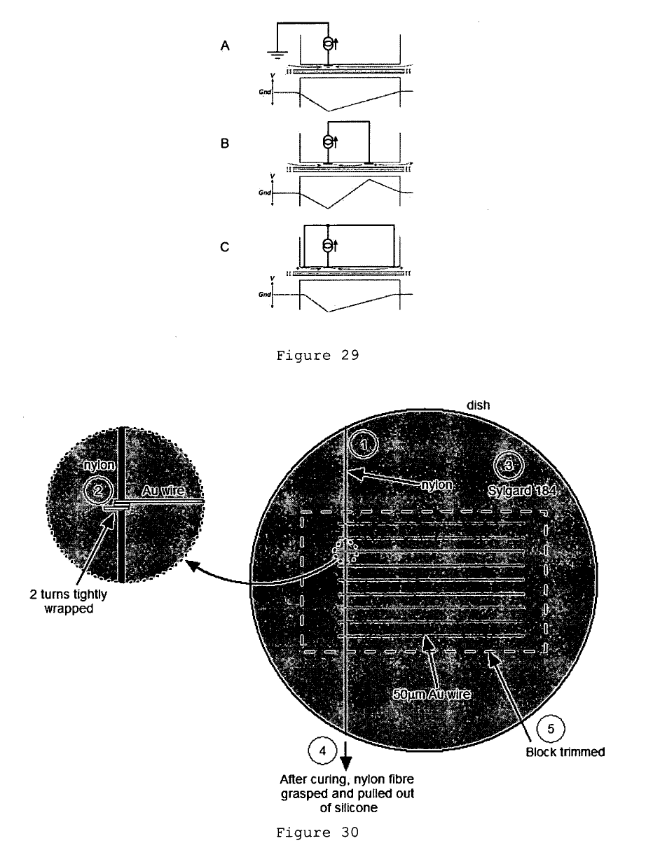

FIG. 30 shows ex vivo device construction

FIG. 31 shows a finished ex vivo device

FIG. 32 shows ex vivo device in use

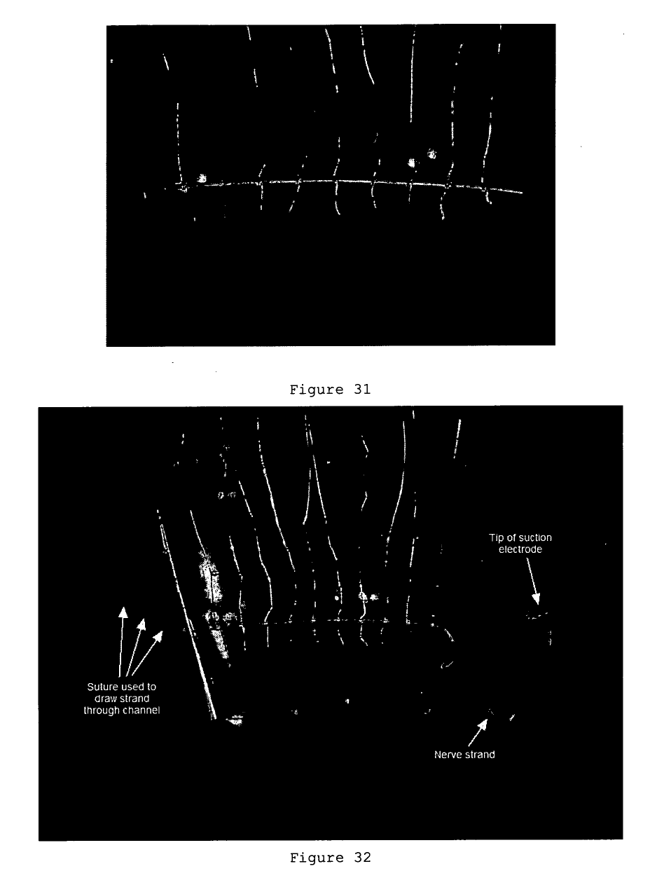

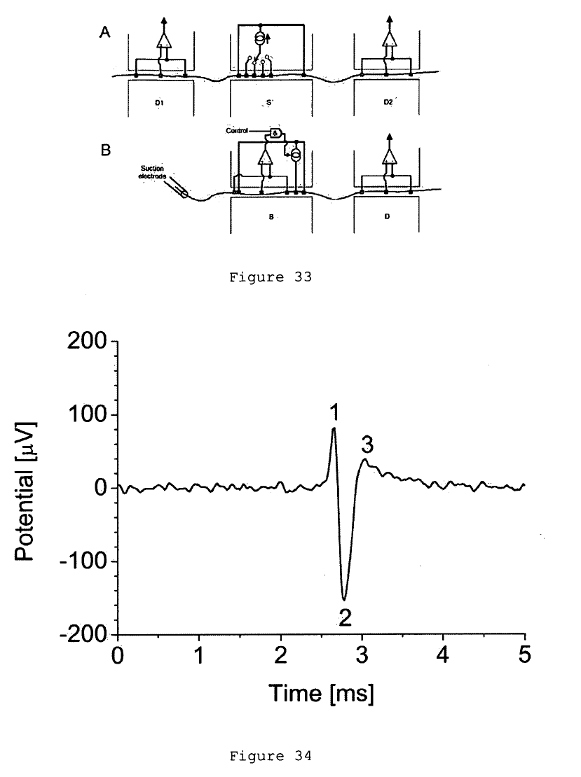

FIG. 33 shows configurations for stimulation and blocking experiments. A: Stimulation experiments, B: Blocking experiment

FIG. 34 shows the form of the extracellular signal recorded as an action potential passes through a microchannel. Phases 1 and 3 occur as the AP enters and leaves the channel respectively. Phase 2 occurs as the AP passes the electrode in the microchannel.

FIG. 35 shows a single unit signal recorded at 1 mm intervals along a 1 cm long, 110 μm diameter channel. The action potential is propagating left to right along the axon. In each trace, the narrow complex at about 0.2 ms is stimulus artefact, while the broader triphasic response at 1 ms is the signal from the resulting action potential

FIG. 36 shows recordings from an electrode at the midpoint of a 10 mm long 110 μm diameter microchannel for 30 nerve strands. Data have been fitted with a straight line and a power curve.

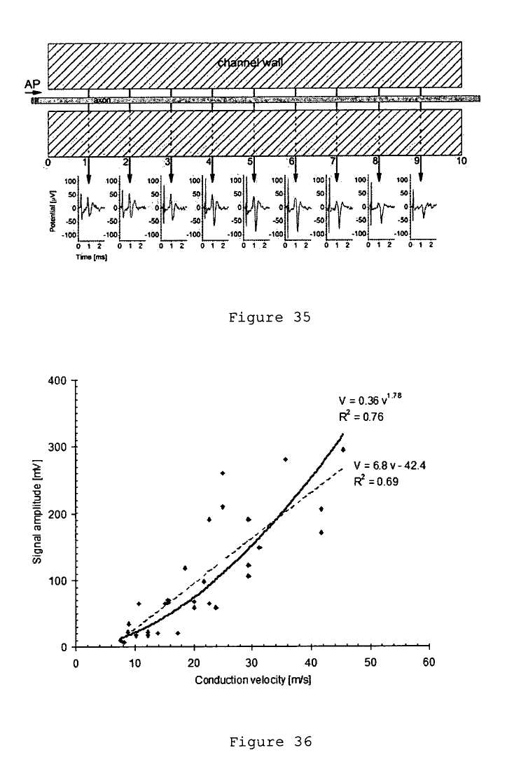

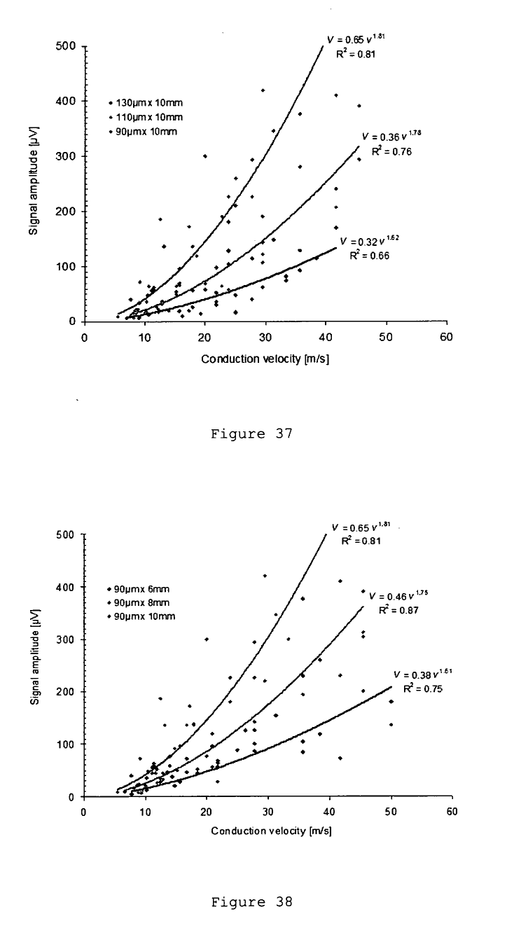

FIG. 37 shows voltage recorded at mid-channel versus conduction velocity for channels of three different diameters

FIG. 38 shows voltage recorded at mid-channel versus conduction velocity for channels of three different lengths

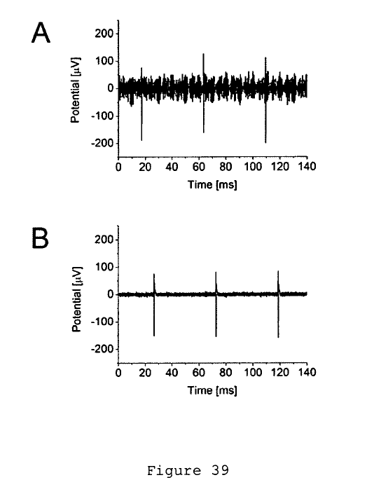

FIG. 39 shows reduction of recording noise. A: Single-ended recording using an electrode at the midpoint of an 8 mm long, 110 μm diameter microchannel referenced to a bath earth. B: Tripolar differential recording between the electrode used in A and two commoned electrodes 1 mm in from either end of the microchannel.

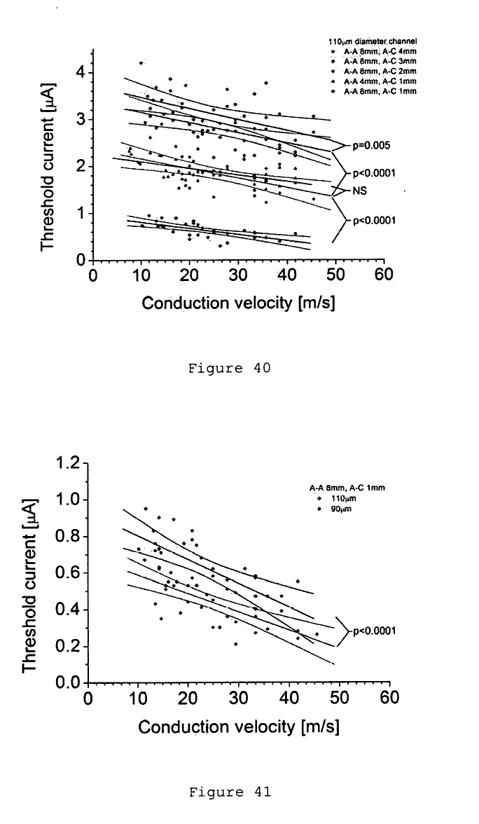

FIG. 40 shows stimulus threshold versus tripolar electrode spacing in a 110 um diameter microchannel. The key shows the separation of the anodes in mm and the distance from the cathode to the nearest anode.

FIG. 41 shows stimulus threshold versus channel diameter. Tripolar configuration with anodes 8 mm apart and cathode 1 mm from nearest anode.

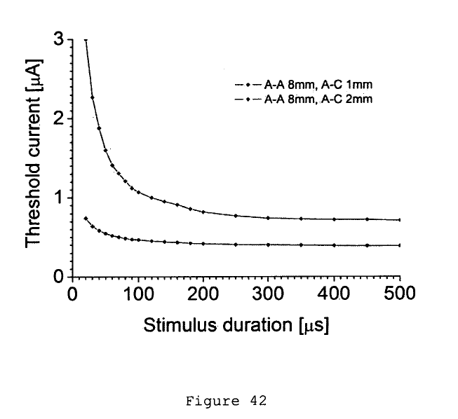

FIG. 42 shows examples of plots of threshold current versus stimulus duration for two tripolar stimulator configurations. Anodes 8 mm apart in both cases. Top trace: cathode 2 mm from nearest anode. Bottom trace: cathode 1 mm from nearest anode. Conduction velocities in the strands featured were 22 m/s for the top trace and 20 m/s for the bottom trace.

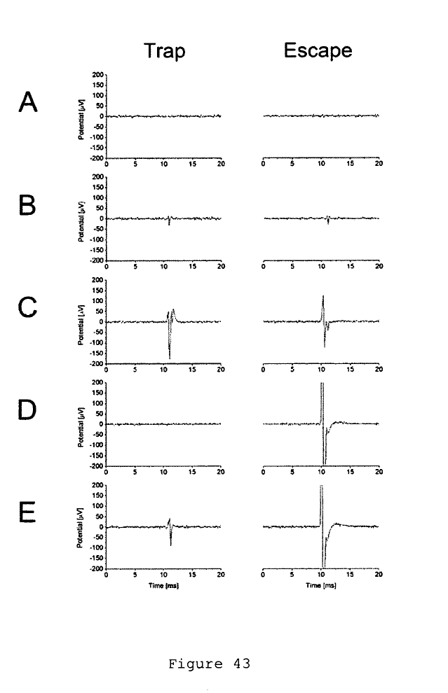

FIG. 43 shows a response to a 400 μs duration stimulus using an asymmetric tripole in a 110 μm diameter microchannel. The cathode is 1 mm away from the ‘trap’ anode and 7 mm from the ‘escape’ anode. The signals above are recorded from the nerve strand either side of the stimulator. Gradually increasing the stimulus current results in the series of changes shown in A-E. A: Low current, no stimulation. B: Single unit stimulation. C: Multiunit activity. D: Extinction of signal beyond the trap anode. E: Anode-break excitation beyond the trap anode.

FIG. 44 shows the three thresholds for a single strand. Lower trace: Threshold for bidirectional stimulation of the first unit to fire. Middle trace: Threshold for suppression of activity passing the ‘trap’ end of the stimulator. Upper trace: Onset of Anode-break excitation. The shaded area is the region where unidirectional stimulation is obtained.

FIG. 45 shows stimulation using an asymmetric tripole (anodes 8 mm apart, cathode 1 mm from nearest anode). Data for 10 strands plus means and 95% ranges. Black squares: Threshold for single-unit bidirectional stimulation. Green circles: Level at which APs completely blocked at trap end. Red triangles: Onset of anode-break excitation.

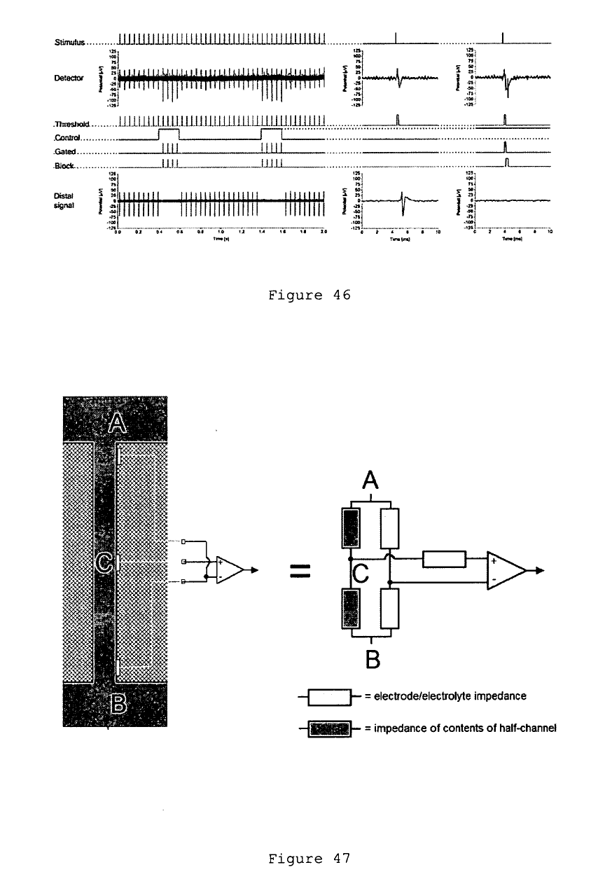

FIG. 46 shows results of the blocking experiment. Stimulus: signal to suction electrode at proximal end of strand; Detector: output from recording tripole in proximal part of microchannel; Threshold: digital signal obtained by applying threshold circuit to the detector signal in the trace above; Control: signal determining whether APs should be blocked or allowed to pass (high=block); Gated: output of AND gate combining ‘threshold’ and ‘control’ signals which passes the pulses in ‘threshold’ only when ‘control’ is high; Block: Stimulating signal to unidirectional stimulator tripole in distal part of microchannel; Distal signal: recording from detector further down the strand. On the left is a 2 second run, while at middle and right are views of single passed and blocked APs respectively.

FIG. 47 shows an example of a recording array with noise reduction through a tripole amplifier circuit.

FIG. 48 shows an example of a recording array with noise reduction through differential amplification of the signal recorded from similarly positioned electrodes in two different channels.

FIG. 49 shows an example of a recording array with noise reduction through differential amplification of the signal recorded from one channel and a reference derived from the signals from a plurality of channels.

### EXPERIMENTS

Every voluntary muscle in the body is supplied by a large number of axons and is capable of a wide range of contractile forces. For example, the muscle flexor digiti minimi, which is responsible for flexion of the little finger and which is just one of several small muscles in the hand, has around 130 axons [5]. Muscles are signalled to contract by action potentials (APs) in these axons. Weak contractions are produced by low frequency activity in a subset of axons, the remaining axons being silent. Gradual increases in force are produced both by increasing the frequency of APs and by activation of increasing numbers of axons (recruitment). There are over thirty muscles in the human forearm and hand effecting movement of the wrist and digits, and most movements use a combination of several of them. Furthermore, the motor fibres in peripheral nerves are outnumbered by other fibres concerned with sensory modalities such as touch, vibration, pain and temperature, feedback information on joint position and muscle stretch, and involuntary vascular musculature which regulates blood flow. This level of complexity stands in stark contrast with that typical of present day experimental devices such as sieve interfaces and needle arrays which typically have from a few tens (sieves) up to a hundred or so electrodes (needle arrays), only a minority of which receive axonal signals without crosstalk from other electrodes [3]. Exactly how many active contacts will be required for satisfactory function in a prosthesis remains an open question. A bare minimum might be to record from one axon for each muscle replaced by the prosthesis (thus 30-40 for a forearm-level upper limb prosthesis). This minimum is far higher than has been achieved reliably with any peripheral nerve interface design.

A travelling action potential (AP) has an amplitude of about 100 mV. Under normal circumstances, most of the AP signal occurs on the inside of the cell, with the potential on the exterior changing much less. This is unfortunate because long-term neural interfaces must rely on purely extracellular recording. The extracellular fluid (ECF) has a low resistivity and occupies a large volume around the axon, and the ionic currents of the AP are small, with the result that the signals recorded outside the axon are typically of the order of 10-500 μV [6].

The membrane voltage Vₘ is defined as Vₘ=Vᵢₙ−Vₒᵤₜ, where Vᵢₙ and Vₒᵤₜ are respectively the voltages on the internal and external surfaces of the axon membrane. At rest Vₘ˜−70 to −80 mV. Consider now two adjacent nodes in a myelinated axon as an AP is going by. Each node depolarises the next by the passage of local currents in a loop as shown in FIG. 1. This current flows because the depolarised node on the left has a more positive membrane potential Vₘ than the resting one on the right. The internal and external currents are equal and opposite; the difference in Vₘ drops across the resistances of the extracellular fluid (R_{ecf}) and the axon (Rₐₓₒₙ) as:

δVₒᵤₜ=iR_{ecf} (1)

and δVᵢₙ=−iRₐₓₒₙ (2)

Dividing (1) by (2) shows that the change δVₘ in Vₘ will be apportioned between changes in Vₒᵤₜ and Vᵢₙ according to:

$$\frac{δ {V}_{out}}{δ {V}_{in}} = - \frac{{R}_{ecf}}{{R}_{axon}} \qquad ( 3 )$$

A range of values is quoted in the literature for axoplasm resistivity (ρₐₓₒₙ). We have used a value of 1.1 Ωm after McNeal [7]. In a 10 μm diameter axon this gives an Rₐₓₒₙ of 4.2MΩ per internode (assuming an internode length of 300 μm). R_{ecf} is a much more difficult quantity to predict. The extracellular environment in a peripheral nerve is composed of interstitial fluid together with space-occupying cellular material including other axons. The extracellular resistivity ρ_{ecf} is therefore inhomogeneous and because of the alignment of the fibres it is also anisotropic [8, 9] being lower longitudinally than transversely. Many authors have followed McNeal's approximation in assuming an isotropic ρ_{ecf} of 3 Ωm, but given that part of the aim of the present study is to examine the decay of signal levels with radial distance from the axon, it is important to reflect this anisotropy and we have therefore specified an axial and a radial resistivity. The values chosen are those used by Veltink et al [10] and are given in table 1.

Another important design criterion for extracellular recording is low instrumentation noise. A metal microelectrode with a typical impedance of 1 MΩ at body temperature (37° C.) in a bandwidth of 10 kHz (enough to record an AP) equates to a thermal (Johnson) noise of around 13 μV rms, or almost 40 μV peak-peak, not all that far away from the extracellular AP signal level. The electrode impedance, and therefore the noise, may be reduced by increasing the electrode surface area using electrode materials such as Platinum Black or Titanium Nitride. Noise due to radiated electromagnetic interference (EMI), including 50 Hz noise coming from the electrical mains or devices connected to it such as switching power supplies also needs to be minimized. Faraday cages and digital noise eliminators may be implemented for laboratory experiments. But in order for the implanted device to be used on a day-to-day basis, screening and filtering techniques must be implemented within the implants in situ. Not only are the extracellular signals small but they also decrease very rapidly with distance away from the axonal membrane. In general, effective transduction requires the fibre to be in close physical contact with an electrode. Furthermore, with myelinated fibres, the transmembrane current is concentrated close to the nodes of Ranvier, stretches of bare axon about 1 μm long, separated by several hundred microns as sketched in FIG. 2. Unless the electrode is positioned close to a node of Ranvier, it is unlikely to pick up anything from the axon concerned. Sieve electrodes are thin, typically 10-20 μm thick, and the tips of needle electrodes are of similar dimension. The chance of a given axon having a node in the plane of a sieve or close to a needle tip is therefore rather small so it is perhaps unsurprising that many electrodes in a sieve or needle array are electrically silent [6, 11].

It is evident from equation (3) that if one could raise R_{ecf} then δVₒᵤₜ should be amplified. From a purely electrical point of view this could be done by altering the composition of the ECF to increase its resistivity (ρ_{ecf}), but in reality this is not a practical proposition since a fairly drastic change in composition would be required to achieve significant amplification, but a normal ECF composition is necessary for continued neuronal viability. The alternative means of raising R_{ecf} is to reduce the ECF volume. This idea was successfully exploited by Huxley and Stampfli in 1949 [12] in their seminal work demonstrating saltatory conduction in myelinated axons, and is utilised to some extent in cuff electrodes. By pulling a teased nerve fibre through a glass capillary of 40 μm diameter Huxley and Stampfli were able to generate an R_{ecf} of 0.5MΩ over 0.5 mm. Later work by Loeb and colleagues [13] explored mathematically the use of short, very narrow tubes to amplify extracellular signals, and suggested that this might produce signals of the order of 1 mV. However this would require axons to grow through holes of 225 μm² cross section (17 μm diameter), which is too small for the largest motor fibres which are of most interest to prosthetic control.

### Methods

Finite element analysis was performed using COMSOL Multiphysics (version 3.2b) running under Microsoft Windows XP on a Dell Precision 380 workstation equipped with a 3.4 GHz Intel Pentium processor and 4 Gb RAM. Models were written in the COMSOL Script language.

An axisymmetric geometry was used as illustrated in FIG. 2. The geometry was drawn in two dimensions in a half-plane (FIG. 2A), and the software was programmed to compute the model solution as if the shapes drawn had been revolved through 360° about the symmetry axis to produce the three dimensional structure shown in FIG. 2B (a cylindrical axon running through a concentric cylindrical channel). Except where explicitly stated, the channel length was 1 cm. The modelled region of axon extended beyond the channel on either side so that an action potential could be observed before, during and after its passage through the channel. Action potentials were triggered by a 0.2 ms, 2 nA current pulse applied to the end of the modelled section of axon.

Separate models were created for unmyelinated and myelinated axons. The unmyelinated model comprised two spaces (the interior of the axon and the extracellular space), both of them filled with a volume conductor (with resistivity ρₐₓₒₙ and ρ_{ecf} respectively) and separated from each other by the axonal membrane which was modelled as a thin film using the Hodgkin-Huxley equations [14]. These equations, originally derived from work with the squid giant axon at 6.3° C., have been adapted for use at 37° C. [15]. The equations are given in the appendix, and model parameters are listed in table 1.

The geometry for the myelinated axon was similar but with the addition of myelin segments along the side of the axon (FIG. 2C). Myelin segment (internode) length in normal (uninjured) nerves varies with fibre diameter but in regenerated nerves it has a relatively constant value of about 300 μm [16]. Since fibres growing through an interface are of necessity regenerating, we have used this value for all sizes of simulated myelinated fibre. The thickness of the myelin layer under normal circumstances also varies with axon diameter, so that the ratio of axon diameter to total fibre diameter (axon and sheath together) is around 0.6-0.7, which is found to be optimal for conduction velocity [17]. This quantity is called the g-ratio.

Regenerated fibres typically have a thinner sheath and thus a higher g-ratio [18]. We have used a value of 0.8 in this model. Node length was 1 μm. There are considerable differences between the membrane of the unmyelinated axon and the nodal membrane of the myelinated axon. Most importantly, the latter lacks voltage activated potassium channels [19]; and while potassium channels are to be found in the adjacent membrane ('paranode') they appear not to play a role in the generation of the AP. The Hodgkin-Huxley model is therefore not appropriate and we have used Sweeney's modification [20] to 37° C. of the model of Chiu et al. [19], which incorporates sodium and leakage currents only. Equations for the myelinated model are given in the appendix and model parameters are listed in table 2.

We have used the same value for transverse and longitudinal ECF resistivities inside the channel as outside it. This is based on the assumption that the micro-channel will become filled with cellular material (axons and Schwann cells) and extracellular matrix broadly similar to that found in the native nerve.

The length of the pre-channel and post-channel sections in the unmyelinated model was 2.5 mm. Because of the much faster propagation speed and the resulting greater spatial spread of each action potential along myelinated fibres, 3 cm of axon was modelled on either side of the channel for these fibres. In both cases this allowed for the disappearance of stimulus artefact from the waveform well before the channel was entered. Models were run for a sufficient period of time to allow propagation of the action potential all the way along the modelled section of axon. For myelinated axons this was up to 3 ms depending on size. Despite the shorter length of modelled axon, unmyelinated models required up to 30 ms because of their much lower conduction velocity. All models were run at a sampling frequency of 50 kHz (most of the power spectrum of the action potential being below 10 kHz).

### Fabrication of Microchannels and Electrodes

An interface was produced consisting of an array of metallic micro-electrodes patterned in microchannels made on and of flexible polyimide films that can be shaped to fit the target nerve. The initial design of the microelectrode device consisted of 5 pairs of electrodes in addition to a common electrode patterned on a 20 μm thick polyimide film (PI 2611, HD Microsystems). The gold electrodes and leads were 60 μm wide, 750 μm apart, and were encapsulated with a −2 μm thick insulating, photosenstitive polyimide layer (Durimide 7005, Fujifilm). Parallel micro-channels (Durimide 7020, Fujifilm) of about 20 μm depth ran across the electrodes. Contact between a certain channel and an electrode took place through a window in the thin insulation layer. The electrodes were connected to the external electronic recording amplifier via contact pads interfaced with an edge connector on one side of the sample, eliminating potential problems concerning bonding or silver gluing contact pads to external connectors. All processing was performed on a 4″ silicon wafer substrate. After complete curing of the polyimide layers, the samples are peeled off from the wafer

After fabrication the micro-channel arrays were sterilized by UV exposure, alcohol cleaning or autoclaving. Then the samples were immersed in a poly-D-lysine solution to absorb PDL onto the polymer surface. The PDL solution was removed and the sample immersed in a laminin solution to allow the laminin to bond to the PDL. The laminin solution was then removed.

DRG (dorsal root ganglion) explants were seeded on the arrays which were immersed in a culture medium (similar composition to extracellular fluid) and kept in an incubator for several days/weeks.

Microchannels with embedded electrodes were fabricated using fine nylon filaments, 50 μm diameter gold wire (Advent Research Materials no. AU517211), and silicone elastomer (Sylgard 184, Dow-Corning). The construction technique is illustrated in FIG. 30 and was as follows. The nylon filaments were suspended taut across 35 mm petri dishes (1) by passing them through small holes made halfway up the sides of the dishes and knotting them securely. At intervals along the filament, one end of a 2 cm long piece of the gold wire was wrapped tightly for 2 turns around the filament (2), the wires then running away perpendicularly. The whole assembly was flooded with the silicone (3), which was cured at 70° C. for 4 hours. During this process the last 5 mm of the free ends of the wires were kept elevated out of the silicone to permit their later external connection. After the silicone had cured, the nylon filament was cut from the sides of the dish, then grasped with a needle-holder and pulled longitudinally out of the silicone (4), thus leaving an empty channel of the same dimensions as the filament with the tightly wrapped gold wire embedded in the silicone forming the channel wall. The silicone block was then trimmed (5) with a razor blade to give a channel of the desired length.

The unencapsulated ends of the gold wires were attached to the bared ends of small single-core wires (Farnell cat no. 119-9108 and similar in different colours) using silver paste. The joints were then encapsulated with more silicone, taking care to keep it away from the channel orifices. Finally, the device was secured to the base of an 85 mm petri dish using a thin layer of silicone poured into the base of the dish, thus forming a bath around the device. A finished device is shown in FIG. 31.

The tight wrapping of the gold wire was intended to exclude the silicone where the wire contacted the nylon filament, so that once the filament was removed, the inner aspect of the wire coil was flush wish the channel wall and exposed to the channel interior. Contact with the channel contents was verified by filling the channel with Hanks Balanced Salt Solution (HBSS, Invitrogen) and measuring the impedance from the bath to the external connection of the wire. Devices containing wires that were open-circuit were discarded and rebuilt.

Several devices were made for different test conditions; the number and position of gold wires along the filament, as well as the filament diameter, varied by application and these details are given below in the relevant sections.

### Obtaining Nerve Tissue

Adult male Sprague-Dawley rats (Charles River, UK) weighing 400-500 g were anaesthetised with an overdose of pentobarbital (Euthatal, Merial Animal Health Ltd) and perfused through the heart with 500 mls of HESS at 4° C., thus exsanguinating and cooling the animal in order to preserve the nerve tissue during the subsequent dissection. Via a long lumbar laminectomy the L5 ventral roots were removed from the spinal cord to the level of the exit foramen, and placed in a dish of HESS at room temperature until used. The roots obtained were approximately 3 cm in length.

### Experimental Conditions

Experiments were performed in HBSS in the baths constructed with each device as described above. The bath was mounted on a warming plate and the temperature of the solution in the bath was continually monitored with a submerged thermometer probe (RS components part no. 427-461). The temperature was maintained within the range 36.5-37.0° C. throughout each experiment.

Placement of Nerve Strands into Channels

The spinal roots were divided by grasping one end with two pairs of No. 5 forceps (Fine Science Tools) and pulling them slowly apart so that the root was peeled into two parts along its length. Each part was split similarly and this process was repeated until a strand smaller than the microchannel was obtained. A short length of very fine (10/0 gauge) monofilament nylon surgical suture (Ethicon) was fed using the same forceps into one end of the microchannel until its tip emerged at the other end. It was then knotted using a single throw around the end of the nerve strand and the suture was used to draw the strand into the channel until it had emerged a few millimetres from the other end (FIG. 32).

### Suction Electrodes

Suction electrodes for stimulation of nerve strands in the recording, blocking and signal transfer experiments were made by drawing out a 5 cm section of 2 mm diameter polyethylene tubing (Portex TUB3670) over a small flame (the pilot flame from a Bunsen burner) and trimming the drawn end to give an aperture of around 100 μm. A silver wire was inserted through the non-flamed end of the tube which was then heat-sealed around the wire. Squeezing the tube with a pair of forceps with the narrow tip in the bath of HESS allowed the expulsion of air and the filling of the electrode on release. The ends of nerve strands sitting in the bath were drawn into the electrode using a similar manoeuvre. Strands were stimulated with a 50 μs square current pulse using a Neurolog NL800A constant current stimulator with its negative terminal attached to the wire in the suction electrode and its positive terminal attached to a counter electrode in the form of a coil of silver wire in the bath.

### Recording Experiments

Three different sizes of filament were used to create channels of diameter 100 μm, 120 μm, and 140 μm, each 10 mm long, to permit investigation of the influence of channel calibre on the recorded signal amplitude. The 100 μm diameter filament was also used to fashion shorter channels (8 mm and 6 mm) to evaluate the effect of channel length. The channels had electrodes at 1 mm intervals along their length.

One end of each nerve strand was pulled through the microchannel, and the other end was stimulated with a suction electrode. Signals from the microchannels were recorded with respect to a silver counter electrode in the bath (FIG. 27A). They were amplified using an AC-coupled amplifier (Neurolog NL104A with headstage NL100AK), passed through a Humbug 50 Hz noise eliminator (Quest scientific), further amplified (Neurolog NL106) then high-pass and low-pass filtered (Neurolog NL144, NL135). Overall voltage gain was ×5000, and the filter corner frequencies were 200 Hz and 10 kHz. The amplified and filtered signal was visualised with a Tektronix TDS2004B digital storage oscilloscope and fed to an analog-to-digital converter (CED Micro 1401 Mk II) sampling at 200 kHz.

Strands of this size will contain several axons, so in order to obtain single-unit activity in each strand, the stimulator was switched on with its output at zero, and then very slowly turned up until the unit with the lowest threshold was activated. As a safeguard, strands were discarded if a second unit was found to be triggerable with up to a 10% increase in stimulus current.

Conduction velocity was measured in each strand in order that it could be used as a surrogate marker for axon diameter. It cannot be satisfactorily measured by examining the time difference between the arrival of the AP at an electrode near the beginning of the channel and subsequently at one further along the channel, for two reasons. First, the form of the signal is predicted to change as the AP progresses down the channel, making valid timing comparisons difficult. Second, the elevation of extracellular impedance due to confinement will cause a mild slowing of conduction velocity, thus creating errors in the estimation of the unconfined velocity and thereby perturbing the relationship with axon diameter. The technique adopted was as follows. The time interval (t₁) from the leading edge of the stimulus pulse to the negative peak of the waveform recorded by the electrode at the midpoint of the microchannel was noted. The strand was then drawn a further 5 mm through the channel and the measurement repeated at the same electrode, yielding a shorter delay t₂. The time difference δt=t₁−t₂ can be estimated satisfactorily because the use of the same electrode ensures the signal will have a constant form. Furthermore, t₁−t₂ corresponds to a 5 mm reduction in the length of the unconfined section of the strand in the bath between suction electrode and channel entrance, rather than the strand in the channel. Conduction velocity is then given simply by v (ms⁻¹)=5/δt, where δt is in milliseconds.

### Noise Reduction

The configuration shown in FIG. 27B was used to evaluate noise reduction using a tripole. A 100 μm, 8 mm long channel was used with a central electrode and electrodes 1 mm in from either end. Recordings were made between the central electrode and the bath, and between the central electrode and the commoned end electrodes.

### Simple Stimulation Experiments

Microchannels for stimulation experiments were fabricated using a similar technique to those used for recording, but instead of a single device, three were fabricated and secured with silicone to the base of the same bath in the arrangement shown in FIG. 33A. Channel S is being characterised as a stimulating device. Channels D1 and D2 serve as detectors to determine when APs are emitted by channel S (this experiment was performed after the recording experiments, so that the principles behind using microchannels as detectors had been validated). For simple (bidirectional) stimulation experiments only S and D1 were used. The strand was drawn into S and D1 in opposite directions by two separate lengths of suture, leaving a loop in the bath between the two devices. Conduction velocity was measured by pulling the strand a further 5 mm through the detector, in a manner analogous with that used in the recording experiments. As with recording, the unit with the lowest stimulation threshold was studied in each strand.

The electronics attached to the detector were exactly as in the recording experiments. For evaluation of the effect of electrode configuration and diameter of channel S on stimulus threshold, 50 μs square current pulses from a Neurolog NL800A constant current stimulator were used. The selector switch allowed the position of the cathode to be moved along the channel. Electrode configurations employed in channel S are described with the results from each test. To investigate the effect of stimulus duration on required stimulus current, the width of the stimulus was adjusted using a Neurolog NL405 pulse width controller.

### Unidirectional Stimulation Experiments

To investigate unidirectional stimulation both detectors D1 and D2 were used, and the selector switch in S was set to the position with the cathode as close as possible to the left anode. Stimulation was with an NL405 pulse width unit set to give a 400 μs pulse and an NL800 constant current source. Current was gradually increased and thresholds noted for (a) the lowest threshold (bidirectional) stimulation, (b) suppression of APs reaching the ‘trap’ end detector, and (c) anode-break excitation.

### Blocking Experiments

In order to investigate whether a microchannel could detect and block an AP in an axon passing through it, a channel was constructed containing a tripolar recording array near one end and an asymmetric tripole for stimulation with its cathode close to the other end (channel B in FIG. 33B). Thus the escape end of the stimulator array is directed back towards the recorder array. A detector D was placed near the stimulator end of the main channel B. After placing a strand through B and D, the end of the strand at the recorder end of B was stimulated with a p suction electrode (in the same fashion as in the recording experiments). The signal from the recorder in B was amplified using the same NeuroLog modules and Humbug noise eliminator as in the experiments above, and fed to a thresholding circuit (NeuroLog NL201 Spike Trigger) which was adjusted to generate a digital pulse sequence where each pulse represented a detected AP. A digital control signal of period 1 sec, duty cycle 20% (generated by the combination of a NeuroLog NL304 period generator and NL405 width unit) was fed, together with the pulse sequence, to an AND gate, which therefore outputted the pulse sequence only when the control signal was high. This gated pulse sequence was used to trigger a 400 μs current pulse delivered to the stimulator in B, and the result was recorded with D.

### Results

### 1.1 Action Potential Modelling

FIG. 3A shows a modelled action potential in a 2 μm diameter unmyelinated axon before it enters the micro-channel. The graph shows the changes in sodium and potassium conductances, and the resulting voltage waveforms. FIG. 3B shows similar curves for a 10 μm diameter axon using the myelinated model.

### 1.2 Extracellular Amplification Due to Confinement

FIG. 4A shows an action potential in a 10 μm myelinated axon approaching a 1000 μm² channel (1 cm before channel entry). Vₒᵤₜ (measured at the axonal membrane) is so small that it hardly deviates from the horizontal axis. FIG. 4B (mid-channel) shows the effect of confinement. There has been a fall in the amplitude of Vᵢₙ, with the appearance of a substantial extracellular signal. Vₒᵤₜ is triphasic; an initial positive phase is followed by a somewhat larger negative phase and finally a smaller positive phase. The central negative phase is coincident with, and opposite in sign to, the peak in Vᵢₙ and represents the expected elevation of the amplitude of Vₒᵤₜ. The peak of Vᵢₙ is correspondingly reduced in height. In all simulations performed, this central negative phase of Vₒᵤₜ had a greater amplitude than the positive phases before or after. Further analysis below will concentrate on this negative phase. The positive phases (which are accompanied by similarly sized pulses of the same sign in Vᵢₙ) result from the abrupt change in R_{ecf} as the AP enters (first positive phase) and then leaves (second positive phase) the channel; in both cases there is a transient net shift of positive charge into the channel. FIG. 4C shows that on emerging from the channel the waveforms return to their original shape.

### 1.3 Effect of Variation of Channel Area

The effect of varying the size of the channel whilst holding the axon size constant is illustrated in FIG. 5. This shows an unmyelinated axon of 2 μm diameter, and a myelinated axon of 10 μm diameter running in several different channel sizes. The fall in the amplitude of the extracellular signal Vₒᵤₜ with increased channel size is entirely to be expected because the increasing size of the volume conductor around the axon results in a decreasing_R_{ecf}.

### 1.4 Effect of Variation of Axon Diameter

FIG. 6 shows the amplitude of Vₒᵤₜ for a range of unmyelinated and myelinated axon diameters in a 1000 μm² channel and illustrates a rapid increase in amplitude with axon size. At the lower diameter end, this increase is due to the fall in Rₐₓₒₙ due to increased intracellular conducting volume, and consequent rise in R_{ecf}/Rₐₓₒₙ. At the higher diameter end, the axon starts to occupy significant amounts of channel space, so that the effect of the decreasing Rₐₓₒₙ is augmented by a simultaneous rise in R_{ecf}.

### 1.5 Effect of Variation of Micro-Channel Length

All of the foregoing analyses have been based on a 1 cm long micro-channel. FIG. 7 shows the effect of different channel lengths from 2 mm to 1 cm in 2 mm increments. The data shown are for a 10 μm myelinated axon in a 1000 μm² cross section channel. There is a monotonic increase in the amplitude of Vₒᵤₜ with increasing channel length, but the rate of increase declines at higher channel lengths.

### 1.6 Radial Potential Distribution

FIG. 8 demonstrates the effect of confinement on the decay of Vₒᵤₜ with radial distance from the centreline of a 10 μm myelinated axon in a 1 cm long, 1000 μm² area channel. For comparison, measurements in both cases are at a node of Ranvier and are taken at the time of the signal peak at each point. The unconfined axon prior to channel entry (FIG. 8A) displays a peak Vₒᵤₜ of 0.1 mV at its surface while for the confined axon at mid-channel (FIG. 8B) Vₒᵤₜ is amplified to over 10 mV (note the break in the vertical axis). The amplitude of Vₒᵤₜ at the channel wall is compared to that at the same radial distance in the unconfined axon. Prior to channel entry, peak Vₒᵤₜ has decayed by 86% at this distance (17 dB). In the channel, there is less than 1% radial attenuation of the signal.

### 1.7 Amplitude Versus Axial Position

FIG. 9 shows the amplitude of Vₒᵤₜ as the axon passes along the channel. In the case of an unmyelinated axon (FIG. 9A, upper trace) the amplitude increases for the first approximately 0.5 mm of the channel, then reaches a plateau which is sustained until a similar distance from the other end of the channel, at which point it falls away again to extra-channel levels. The myelinated axon (FIG. 9A, lower trace) behaves differently: the amplitude of Vₒᵤₜ never reaches a plateau, but rather increases gradually to a maximum, then turns and falls away gradually towards the channel exit. Note that the maximum amplitude is not at the centre of the channel but about two thirds of the way along it. The maximum amplitude of Vₒᵤₜ is 13 mV, which when compared to a value of 100 μV (at the nodes) prior to channel entry gives a gain of greater than 100.

### 1.8 Relationship to Node Position

FIG. 9B shows a close-up of a 1 mm section of the same 10 μm myelinated axon before it enters the channel. The amplitude of Vₒᵤₜ is low and heavily dependent upon axial position. Contrast this with FIG. 9A: not only is the amplitude of Vₒᵤₜ much greater once the axon is in the channel, but in addition the influence of node position has disappeared (there are 33 nodes within the length of the channel; note the different horizontal and vertical scales in FIGS. 9A and 9B).

### 1.9 Conduction Velocities

The elevated R_{ecf} would be expected to cause a decrease in conduction velocity by increasing the time constant for local membrane capacitance charging/discharging (proportional to Cₘ(R_{ecf}+Rₐₓₒₙ)). Table 3 gives the conduction velocities for a range of axon and channel size combinations, both myelinated and unmyelinated. Whilst the exact values for velocity will depend heavily on the model used and its parameters, any errors due to these factors will be systematic and the trends shown in the table will still hold. The main point to note is that for the myelinated fibres (which are of greatest interest to prosthetic control) the conduction delay due to transit time through the micro-channel is under a millisecond in all cases.

Our study demonstrates that confining axons in micro-channels provides amplification of the extracellular component of the action potential. The recordable extracellular signal Vₒᵤₜ increases rapidly with axon diameter and decreases with increasing channel cross section. The pattern of extracellular voltage change recorded in micro-channels differs between myelinated and unmyelinated axons. The slow propagation velocity in unmyelinated axons means that the AP occupies a short stretch of the axon (the model predicts about 0.5 mm for a 1 μm unmyelinated axon), and so the extracellular signal amplitude plateaus a short distance into the channel and remains constant until it approaches within a similar distance of the other end. Myelinated axons display a much faster conduction velocity and as a result the AP is spread over a much longer section of axon (around 2.5 cm for a 10 μm myelinated axon). This is reflected in the fact that the amplitude of the extracellular signal does not plateau but progressively increases along the channel before turning and decreasing towards the exit. The turning point is not located at the centre of the 1 cm channel as one might expect, but approximately two thirds of the way along it (7 mm); the amplitude at the midpoint (5 mm) is about 85% of the maximum. Clearly the optimal position for electrode placement is where the signal is largest, and simulation provides a way to estimate the ideal location for any particular geometry. A much longer channel would certainly allow the signal to grow further; one would expect it to reach maximum when the length of channel both before and after the electrode(s) was greater than the spatial spread of the AP, but this would require a channel several centimetres long which would probably be impractical in most interface designs.

The extracellular voltage is nearly constant transversely across the channel: this suggests that it may not be necessary to bind the axon to the electrode surface. The recording should be similar whether the axon sits directly on the surface of an electrode in the channel wall, or over on the opposite side of the channel. Even more interestingly, in the myelinated case the amplitude of Vₒᵤₜ changes smoothly along the channel with no evidence of dependence on position with respect to nodes of Ranvier. Thus the micro-channel structure frees us from (i) the need to bind the axon to the electrode, and (ii) the worry of getting the electrode near to a node of Ranvier. According to our model, any electroded micro-channel which contains an axon should be functionally active.

In order to ensure fibre selectivity, the micro-channels should be made of a dielectric material. With insulating channel walls, and signal amplification dropping to unity at the end of the channel, it should not be possible for the amplified signal to be picked up by an electrode in an adjacent channel.

The axons of most interest for driving limb prostheses are those which control voluntary movement, i.e. the largest myelinated fibres. The ability to discern signals in these axons from those in small unmyelinated fibres running in the same channel is important. Unmyelinated and myelinated axons of similar diameter produce comparable amplified voltages (see FIG. 6). Fortunately, the size differential between myelinated and unmyelinated fibres has a highly beneficial effect on the relative amplitude of Vₒᵤₜ so that for example the amplitude of Vₒᵤₜ for a 10 μm myelinated axon in a 1000 μm² channel is over 25 times higher than that for a 2 μm unmyelinated axon in the same position. Thus fibre type selectivity in a single micro-channel could potentially be accomplished with a simple thresholding circuit. It is important to note that when large axons are cut and regenerate, the diameter of the regenerated part is initially considerably smaller than the original, and they later gradually enlarge [21]. This suggests that recorded signals at first will be small and fibre type selectivity will initially be relatively poor, but both of these can be expected to improve with time as the fibres grow.

In conclusion, micro-channels are provide significant amplification of extracellular axonal signals, freedom from the requirements to bind axons to electrodes or position electrodes close to nodes of Ranvier, a high proportion of functionally active channels, low crosstalk, and good fibre-type selectivity.

### 2. Experimental Tests Ex Vivo

A prototype interface was made comprising one enclosed channel with several microelectrodes in the channel. The prototype was prepared on small glass substrate (10 mm×12 mm). A metallic stack of 8 nm thick chromium (adhesion layer) and of 175 nm thick gold was patterned as electrodes+leads+contact pads. The micro-channel was prepared by casting silicone elastomer on a several cm long, 70 um wide, ˜120 um high line made of SU8 (a thick epoxy based photoresist) on silicon wafer. The SU8 line was treated with a release layer prior silicone casting for easy removal of the silicone structure. The moulded silicone channel was peeled off the wafer. Then the gold electrode array on glass and the bottom surface of the silicone mould were exposed to a 45 sec air plasma, and stuck together. This procedure enables irreversible bonding between silicone and glass. Note that the channel had to be carefully aligned across the gold electrodes.

The electrode contact pads were soldered to individual wires (to be connected to the electronic hardware).

The device (except the channel) was flooded with a transparent and electrical insulating elastomer to ensure the insulation of the device (the electrodes are only exposed to the saline solution in the micro-channel).

The channel was filled with physiological saline. A nerve strand of about 50 micrometres diameter was peeled from the L5 dorsal root in an anaesthetised rat, detaching it proximally from the spinal cord but leaving it attached distally to the L5 spinal nerve. The strand was inserted into the micro-channel by passing a fine (30 μm) nylon fibre through the channel, tying it to the free proximal end of the nerve strand, and using it to draw the nerve strand into the channel. The entire setup was flooded with physiological saline. Outside of the channel, an electrode made from silver wire was brought into contact with the nerve strand. Action potentials were generated in the strand by mechanically stimulating the receptive field of the axons contained in the strand (located on the hindlimb). No signal was recorded with the electrode situated outside the channel. However, a clear signal of ˜0.1 mV was recorded from the electrodes near the centre of the channel. The signal was much smaller in electrodes closer to the ends of the channel. FIG. 24 shows action potentials recorded with this setup.

The effect of transparency on the proportion of axons entering the microchannels was investigated. It was observed that the proportion of axons entering the microchannels was directly related to the transparency of the interface (FIG. 25). The higher the transparency, the higher the observed number of entering axons in the device.

### 3 Recording

### 3.1 Shape of the Recorded Signal

A typical example of the signal recorded at the midpoint of a microchannel is shown in FIG. 34. The waveform is triphasic, a pattern which is seen in recordings from cuffs. The initial positive phase occurs as the AP crosses the entrance to the channel, the negative peak occurs as it passes the recording electrode, and the final positive phase occurs as it crosses the exit from the channel.

3.2 Signal Amplitude with Respect to Longitudinal Position

FIG. 35 shows the signal recorded at 1 mm intervals from a 10 mm long, 110 μm diameter channel. As predicted, the signal is relatively small near the channel entrance, increases to a maximum near the midpoint of the channel, then falls away again towards the exit.

3.3 Signal Amplitude with Respect to Axon Diameter

FIG. 36 shows the results of measurements on 30 strands in a 110 μm diameter 10 mm long channel. The amplitude of the recorded signal (negative phase) from a mid-channel electrode is plotted against the conduction velocity (surrogate for axon diameter). There is a clear positive correlation between signal amplitude and conduction velocity, and therefore between signal amplitude and axon diameter.

The question of what type of function to try to fit the data with is not trivial. As a first approximation, Rₐₓₒₙ should be inversely proportional to the cross-sectional area of the axon, i.e. inversely proportional to the square of the axon diameter φₐₓₒₙ:

Rₐₓₒₙ∝1/φ²ₐₓₒₙ (7)

φₐₓₒₙ is the diameter of the axon not including myelin. As previously noted, conduction velocity v is proportional to fibre diameter (Hursh 1939) with a constant of proportionality of around 6 ms⁻¹ per μm, but in this case ‘fibre diameter’ (φ_{fibre}) includes the myelin sheath:

v=6φ_{fibre} (8)

The two diameters φₐₓₒₙ and φ_{fibre} are related by:

φₐₓₒₙ=gφ_{fibre} (9)

The so-called ‘g-ratio’ in normal nerves is around 0.6-0.7, but it is not constant and decreases with increasing axon diameter. A more accurate relationship calculated from studies of rat sciatic nerve (Birren and Wall 1956) is:

φ_{fibre}=2.38φₐₓₒₙ^{0.73} (10)

Using (10) to substitute for φ_{fibre} in (8):

v=kφₐₓₒₙ^{0.73} (11)

. . . where k is a constant of proportionality. Using this to substitute for φₐₓₒₙ in (7) and re-arranging:

Rₐₓₒₙ=k′v^{-2.8} (12).

. . . where k′ is another constant. Putting this in (6) we get:

$${V}_{out}^{max} = - \frac{{R}_{ecf}}{{R}_{ecf} + {k}^{′} {v}^{- 2.8}} {V}_{AP} \qquad ( 13 )$$

Despite the fact that we are elevating R_{ecf} by confining the axon in the microchannel, Rₐₓₒₙ remains large by comparison, so we have:

However in practice such a steep relationship cannot be expected because as has already been discussed, equation (6) only holds for APs with a spatial spread less than the microchannel length. Vₒᵤₜ developed by APs that are spatially longer than this will be attenuated, and increasingly so as a progressively smaller part of the AP fits into the channel. This will decrease the steepness of the slope. Nevertheless it seems reasonable to attempt to fit a power curve to the data. This has been done in the figure, and as expected the index has reduced, from v^{2.8} to v^{1.78}. This is in remarkably good agreement with a mathematical model of cuffs which predicted a power relationship with an index of 1.8 (Struijk 1997), and recordings from cuffs in the cat giving an index of 1.7 (Stein et al. 1977).

3.4 Signal Amplitude with Respect to Channel Diameter

FIG. 37 reproduces the data of the previous figure and also shows measurements taken in microchannels of the same length (10 mm) but smaller and larger calibers (90 μm and 130 μm). There are 30 data points in each series. As expected, narrower channels produce higher signal amplitudes. Each series has been fitted with a power curve as above. The index of v in the 90 μm series is similar to that in the 110 μm series, while in the 130 μm series it is slightly lower at 1.62.

3.5 Signal Amplitude with Respect to Channel Length

FIG. 38 shows recordings in 3 microchannels of the same diameter but different lengths (6 mm, 8 mm, and 10 mm, 30 strands in each). Curves have been fitted to each dataset. It is clear that longer channels produce higher recordable signals. Once again the index of v is in the range 1.6 to 1.8.

3.6 Noise Reduction with Tripolar Configuration

FIG. 39 shows single-unit recordings taken from a strand in an 8 mm long, 110 μm diameter microchannel. Pane A shows the signal recorded using an electrode at the midpoint of the channel referenced to earth (the HESS bath) and a single-ended amplifier. The signal in Pane B has been recorded differentially between the same central electrode and two commoned electrodes 0.5 mm from each end of the channel.

The amplitude of the signal in pane B is slightly lower than that in A, consistent with the effective reduction in length of the recording device. However, the differential recording has reduced the noise level dramatically. The root-mean-square noise voltage in this differential recording is 13% of that in single-ended mode, a reduction of 17 dB.

### 4 Stimulation

### 4.1 Stimulation Thresholds, Axon Size and Device Asymmetry

FIG. 40 shows the current required for stimulation using a tripolar electrode configuration with a 50 μs pulse in a 110 μm diameter microchannel. The black, red, green and blue points were all recorded with anodes spaced 8 mm apart. With each group is a fit line (solid) together with its 95% confidence bands (dashed). There is a downward trend in stimulus current with conduction velocity in all groups but this trend is modest compared to the dramatic changes with axon size that were seen in recording. The difference between the groups is the position of the cathode. The black group represents the symmetrical case, with the cathode 4 mm from either anode. Bringing the cathode closer to one of the anodes (red: 3 mm; green: 2 mm; blue: 1 mm) progressively reduces the threshold current (analysis of covariance showed that all groups were significantly different). Thus the greater the asymmetry of the tripole, the lower the current required for stimulation. This is understandable because increasing asymmetry leads to progressively more of the current being passed between the cathode and the anode to which it is closest, generating a steeper potential gradient in the channel.

The purple data points and fit line were produced using a tripole with the same proportions as the green group but scaled down by half (i.e. 4 mm from anode to anode, with the cathode 1 mm from the nearest anode). The purple fit line lies well within the confidence bands of that for the green group, and analysis of covariance confirms that the distributions of green and purple points are not significantly different. The scaling therefore appears to have no significant effect on the threshold current as long as the proportions are preserved. This is again understandable, because scaling the array whilst maintaining its proportions will not affect the current flowing between cathode and either anode so that the potential gradient generated is of the same steepness.

### 4.2 Stimulation Thresholds and Channel Diameter

FIG. 41 reproduces the lowermost dataset from the previous graph (blue) and a further group has been added (green) showing thresholds using a tripole of the same dimensions in a narrower channel (90 μm). As expected, the narrower channel results in significantly reduced stimulus current requirements.

### 4.3 Stimulus-Duration Curves

FIG. 42 shows stimulus-duration curves for individual strands in two tripole configurations (anodes 8 mm apart, cathode 1 mm from nearest anode in lower trace and 2 mm in upper trace). The stimulus current is lower for the more asymmetric configuration at all stimulus durations. Rheobase for the lower trace is 54% of that for the upper trace.

### 4.4 Unidirectional Stimulation

FIG. 43 shows the output from the detectors either side of a tripolar stimulating channel as the stimulator delivers progressively increasing current levels (400 μs duration square pulse). The cathode is 1 mm away from the ‘trap’ anode and 7 mm from the ‘escape’ anode. At low currents there is no stimulation (A). Increasing current generates first single-unit activity (B) then multi-unit activity (C). Further increasing the current prevents the APs reaching the detector at the ‘Trap’ end, while allowing them through to the detector at the ‘Escape’ end, thus demonstrating unidirectional stimulation. Eventually APs reappear at the trap end detector. These occur around 300 μs later than the AP in B, in keeping with anode-break excitation at the end of the stimulus pulse (although the stimulus is 400 μs long, the trap end anode is closer to the detector than the cathode, reducing the difference in timing between the signals in B and E).

FIG. 44 shows the thresholds for (a) activation of the first unit (bidirectionally), (b) suppression of activity at the ‘trap’ end, and (c) onset of anode-break excitation, for a single strand in a 110 μm diameter channel over a wide range of stimulus durations. The hatched area represents the combinations of pulse length and current that can be used to obtain unidirectionally propagating APs. Below 350 μs this window progressively narrows, and at short durations unidirectional stimulation is not possible.

FIG. 45 shows measurements on 10 strands using 400 μs stimulus pulses. In each case the lowermost point is the stimulus threshold of the first single unit to fire as the stimulus current is increased. The middle point is the level at which APs are suppressed at the trap end of the tripole and thus unidirectional stimulation is achieved. The uppermost point is the current at which anode-break excitation occurs. On the extreme right are the mean and 95% range for each of these quantities. Importantly, the ranges for unidirectional stimulation and anode-break excitation do not overlap. Thus with a current in the region of 5 μA it should be possible to produce unidirectional stimulation whilst avoiding ABE in nearly all cases.

### 5. Blockade

FIG. 46 is a timing diagram showing the results of the blocking experiment. On the left is a 2-second recording from various points in the circuit. The top trace ('Stimulus') shows the input to the suction electrode. The next trace ('Detector') shows the resulting train of action potentials being recorded from the proximal end of channel B. This is converted to a pulse sequence ('Threshold') which is then ANDed with the ‘Control’ signal to produce ‘Gated’ where the pulse sequence is present only when ‘Control’ is high. This causes the stimulator in channel B to output unidirectional stimulation pulses ('Block'). As can be seen from the bottom trace, this has extinguished the APs in the distal axon while ‘Control’ is high.

We have shown that confinement of axons in insulating microchannels, and thus restriction of ECF volume, amplifies the recordable extracellular signal generated by propagating action potentials.

The recorded signal amplitude increases with (i) decreasing channel diameter, (ii) increasing channel length, and (iii) increasing AP conduction velocity, which is a surrogate marker for axon diameter. The signal is small near the channel entrance, increases to a maximum and then falls away again towards the channel exit. Substantial noise reduction can be achieved with differential recording using a tripolar electrode configuration.

The range of conduction velocities seen fits well with published data. Myelinated fibres in the rat range up to around 12 μm (Schmalbruch 1986) (this is total fibre diameter including myelin; with a normal g-ratio this corresponds to an axon diameter of about 8.5 μm). Fibre diameters and conduction velocities in humans range up to around twice those seen here, and the trend seen in the results described above provides indication that the recorded signals in the largest alpha motor fibres would be considerably higher than those we have recorded.

Whilst the positions of the nodes of Ranvier in the active axon in the strand under test are unknown, two lines of evidence demonstrate that confinement in the channel is having the desired effect of making the recorded signal independent of node position. First, the signal is recordable at all electrode sites along the channel. It is highly unlikely that a node would happen to be aligned with each and every one of the nine electrodes in the channel. Second, if the signal was restricted to the region of the node, one would expect that a signal present initially, which would have to be from an axon with a node close to the electrode, would disappear after the 5 mm pull-through unless the strand happened to come to rest with another node close to the electrode, which ought to be an infrequent occurrence. In all the strands examined this never occurred; indeed the signal was observed to be present and undiminished throughout the pull-through process.

This is an important result, because in an interface it is obviously desirable that recording can be obtained from as many fibres as possible, not just those that happen to have nodes in the proximity of the electrode. It is not obvious how Schwann cells could be persuaded to myelinate regenerating fibres within an implant in such a way as to ensure that nodes of Ranvier formed opposite fixed electrodes built into the device. This study shows that microchannel confinement circumvents this difficult biological problem.

The rapid rise in recorded signal amplitude with axon diameter means that the signals which we are most interested in recording for prosthetic motor control, conveyed in the largest (Aα) fibres, will be the largest, and comfortably so. Each microchannel in an interface may contain a group of a few tens of axons, and this finding means that simple thresholding circuitry could be used to listen selectively from motor fibres, while rejecting the background ‘chatter’ in other (smaller) axons. The thresholding approach allows motor signals to be picked out from the electrical traffic in a mixed fibre-type channel. Once again, a potentially difficult biological problem is avoidable by careful exploitation of the electrical properties of the device.

Microchannel confinement has also been shown to be highly beneficial for stimulation. In situations where bidirectional stimulation is acceptable, the required current and charge transfer are extremely small. Stimulus threshold decreases slowly with axon diameter, opening up the possibility of a degree of selectivity for stimulation of large fibres, although the dependence on diameter is not as strong as for recording.

Whilst increasing channel length improves recorded signal amplitude, for stimulation the important parameter is the relative electrode positions, specifically the degree of asymmetry in the tripolar configuration we have used. The configurations shown by the data points in green and purple in 3 gave statistically identical results despite the former being twice as long as the latter. Shorter devices may better support axon regeneration, and reduce the voltage required from the stimulator for a given current, thus reducing power consumption. Substantially smaller lengths may not work well because we require that there are at least two nodes in the high-gradient region between the cathode and its closest anode, which means that this region must be longer than the internode.

Unidirectionally propagating APs may also be generated, although this requires somewhat higher currents for longer periods. Unidirectional stimulation is an important ability for three reasons. Firstly, it may allow modality-specific stimulation. In a mixed fibre-type channel, it could generate purely centrally-directed or purely peripherally-directed action potentials. The peripherally-directed signals would activate muscles if they are in motor fibres, while having no effect in sensory axons, so purely motor stimulation would result. Conversely the centrally-directed signals would be effective in sensory axons but not in motor axons, thus producing purely sensory stimulation. Secondly, the presumption routinely made in designing FES systems, and repeated above, that retrograde signals are harmless may not be entirely correct. Antidromic APs reaching large alpha motor cells in the spinal cord can result in ‘reflection’ of some APs back towards the periphery, evoking a weak delayed muscle contraction visible in the EMG as the F-wave. It is not clear whether this would be clinically significant. Thus, even if we find some means of segregating axons into channels of one particular type, it may still be desirable to stimulate the ‘motor’ channels unidirectionally to prevent the generation of the F-wave. Thirdly, unidirectional stimulation is a necessary building block for collision blockade.

In cuffs, there may be no clear window between the current required for unidirectional stimulation and that which causes anode-break excitation, i.e. the equivalent of the hatched region in Figure does not exist. This is likely to be because the stimulating field is non-uniform, so that a stimulus large enough to cause unidirectional stimulation of some axons has already gone beyond the level required to cause ABE in others where the current density is higher. The more uniform current within a microchannel is shown here to open up a clear window between unidirectional stimulation and ABE, even with a square-wave stimulus. A quasitrapezoidal pulse might widen this window further.

The blockade experiment shows that a microchannel together with suitable circuitry can automatically detect an action potential entering it and block it so that it does not emerge from the other end. This is a potentially useful facility in an interface used in the nerve injury (rather than amputation) scenario, where misrouting of regenerated fibres has occurred. The dysfunctional passage of APs via a misrouted axon to the ‘wrong’ muscle could be blocked by the microchannel through which that axon passes, and if desired the blocking circuitry could also trigger a stimulator in a different channel to reroute impulses to the ‘right’ muscle.

### REFERENCES

[1] C. S. Lovchik and M. A. Diftler, “Compact dexterous robotic hand,” U.S. Pat. No. 6,244,644
[2] K. W. Horch and G. S. Dhillon, Neuroprosthetics Theory and Practice. Utah: World Scientific Publishing, 2002.
[3] A. Ramachandran, M. Schuettler, N. Lago, T. Doerge, K. P. Koch, X. Navarro, K. P. Hoffman, and T. Stieglitz, “Design, in vitro and in vivo assessment of a multichannel sieve electrode with integrated multiplexer,” Journal of Neural Engineering, vol. 3, pp. 114-124, 2006.
[4] L. Wallman, A. Levinsson, J. Schouenborg, H. Holberg, L. Montelius, N. Danielsen, and T. Laurell, “Perforated silicon nerve chips with doped registration electrodes, in vitro performance and in vivo operation,” IEEE Transactions in Biomedical Engineering, vol. 46, pp. 1065-1074, September 1999.
[5]H. Santo Neto, V. C. de Carvalho, and M. J. Marques, “Estimation of the number and size of human flexor digiti minimi muscle motor units using histological methods,” Muscle and Nerve, vol. 21, pp. 112-114, 1998.
[6] A. F. Mensinger, D. J. Anderson, C. J. Buchko, M. A. Johnson, D. C. Martin, P. A. Tresco, R. B. Silver, and S. M. Highstein, “Chronic recording of regenerating VIIIth nerve axons with a sieve electrode,” Journal of Neurophysiology, vol. 83, pp. 611-615, 2000.
[7] D. R. McNeal, “Analysis of a model for excitation of myelinated nerve,” IEEE Transactions in Biomedical Engineering, vol. 23, pp. 329-337, 1976.
[8] I. Tasaki, “A new measurement of action currents developed by single nodes of Ranvier,” Journal of Neurophysiology vol. 27, pp. 1199-1206, 1964.
[9] K. W. Altman and R. Plonsey, “Analysis of the longitudinal and radial resistivity measurements of the nerve trunk,” Annals of Biomedical Engineering, vol. 17, pp. 313-324, 1989.
[10] P. H. Veltink, B. K. Van Veen, J. Struijk, J. Holsheimer, and H. B. K. Boom, “A modelling study of nerve fascicle stimulation,” IEEE Transactions in Biomedical Engineering, vol. 36, pp. 683-692, 1989.
[11] A. Branner and R. A. Norman, “A multielectrode array for intrafascicular recording and stimulation in sciatic nerve of cats,” Brain Research Bulletin, vol. 51, pp. 293-306, 2000.
[12] A. F. Huxley and R. Stampfli, “Evidence for saltatory conduction in peripheral myelinated nerve fibres,” Journal of Physiology, vol. 108, pp. 315-339, 1949.
[13] G. E. Loeb, W. B. Marks, and P. G. Beatty, “Analysis and microelectronic design of tubular electrode arrays intended for chronic, multiple single-unit recording from captured nerve fibres,” Medical and Biological Engineering and Computation, vol. 15, pp. 195-201, 1977.
[14] A. L. Hodgkin and A. F. Huxley, “A quantitative description of membrane current and its application to conduction and excitation in nerve,” Journal of Physiology, vol. 117, pp. 500-544, 1952.
[15] F. Rattay and M. Aberham, “Modelling axon membranes for functional electrical stimulation,” IEEE Transactions in Biomedical Engineering, vol. 40, pp. 1201-1209, 1993.
[16]H. B. Hiscoe, “Distribution of nodes and incisures in normal and regenerated nerve fibres,” The Anatomical Record, vol. 99, pp. 447-475, 1947.
[17] R. S. Smith and Z. J. Koles, “Myelinated nerve fibers: computed effect of myelin thickness on conduction velocity,” American Journal of Physiology, vol. 219, pp. 1256-1258, 1970.
[18] J. M. Schröder, “Altered ratio between axon diameter and myelin sheath thickness in regenerated nerve fibres,” Brain Research vol. 45, pp. 49-65, 1972.
[19] S. Y. Chiu, J. M. Ritchie, R. B. Rogart, and D. Stagg, “A quantitative description of membrane currents in rabbit myelinated nerve,” Journal of Physiology, vol. 292, pp. 149-166, 1979.
[20] J. D. Sweeney, J. T. Mortimer, and D. Durand, “Modelling of mammalian myelinated nerve for functional neuromuscular electrostimulation,” in 9th Annual Conference of the IEEE Engineering in Medicine and Biology Society, Boston, Mass., 1987, pp. 1577-1578.
[21] S. Ramon y Cajal, Degeneration and regeneration of the nervous system vol. 1. London: Oxford University Press, 1928.
[22] N. Lago, D. Ceballos, F. J. Rodriguez, T. Stieglitz, and X. Navarro, “Long term assessment of axonal regeneration through polyimide regenerative electrodes to interface the peripheral nerve,” Biomaterials, vol. 26, pp. 2021-2031, 2005.

### ADDITIONAL REFERENCES

Ballestrasse C L et al. 1985. Ann Biomed Eng 13(5):405-24.
Birren J E et al 1956. J Comp Neurol 104(1):1-16.
Branner A et al. 2000. Brain Research Bulletin 51:293-306.
Branner A et al. 2004. IEEE Trans Biomed Eng 51(1):146-57.
Della Santina CC et al 1997. J Neurosci Methods 72(1):71-86.
Edell D J. 1986. IEEE Trans Biomed Eng 33(2):203-14.
Fodstad H. 1989. Stereotact Funct Neurosurg 53(4):209-22.
Gaunt R A et al 2006. Prog Brain Res 152:163-94.
Grill W M et al 1998. IEEE Trans Rehabil Eng 6(4):364-73.
Hansen M et al 2004 IEEE Trans Neural Syst Rehabil Eng 12(1):81-8.
Heiduschka P et al 1998. Prog Neurobiol 55(5):433-61.
Hoffer J A et al 1974. Society for Neuroscience. p 258.
Hursh J B. 1939. American Journal of Physiology 127(1):131-139.
Huxley A F et al 1949. Journal of Physiology 108:315-339.
Inmann A et al 2004. Med Eng Phys 26(6):449-58.
Karlsson M et al 1996. J Comp Neurol 364(2):211-8.
Kovacs G T et al 1992. IEEE Trans Biomed Eng 39(9):893-902.
Lago N et al 2005. Biomaterials 26:2021-2031.
Lefurge T et al 1991. Ann Biomed Eng 19(2):197-207.
Malagodi M S et al. 1989. Ann Biomed Eng 17(4):397-410.
Mensinger A F et al 2000. Journal of Neurophysiology 83:611-615.
Merrill D R et al 2005. J Neurosci Methods 141(2):171-98.
Morton S L et al (1994. J. Electrochemical Society 141(1):122-130.
Navarro X et al 2005. J. Peripheral Nervous System 10:229-258.
Nicholls J G et al 1992. Sunderland, Mass.: Sinauer Associates. p 467-518.
Ramachandran A et al. 2006. Journal of Neural Engineering 3:114-124.
Ramon y Cajal S. 1928. Degeneration and regeneration of the nervous system. London: Oxford University Press.
Rutten W L C. 2002. Annual Reviews in Biomedical Engineering 4:407-452.
Rutten W L C et al 1995. Biosensors and Bioelectronics 10:141-153.
Rutten W L C et al 1991. IEEE Transactions in Biomedical Engineering 38:192-198.
Santo Neto H et al 1998. Muscle and Nerve 21:112-114.
Schachter S C, Schmidt D M D. 2003. Vagus nerve stimulation. London:
Martin Dunitz. x, 173 p. p.
Schmalbruch H. 1986. Anat Rec 215(1):71-81.
Stein R B et al 1975. Canadian J. of Neurological Sciences 2:235-244.
Stein R B et al 1977. Brain Res 128(1):21-38.
Stieglitz T. 2004. Electrode materials for recording and stimulation. In: Horch K W, Dhillon G S, editors. Neuroprosthetics Theory and Practice. Singapore: World Scientific Publishing Co.
Struijk J J. 1997. Biophys J 72(6):2457-69.
Sweeney J D et al 1990. IEEE Trans Biomed Eng 37(7):706-15.
Tyler D J, Durand D M. 2002. IEEE Trans Neural Syst Rehabil Eng 10(4):294-303.
van den Honert C et al 1979. Science 206(4424):1311-2.
Wallman L et al 1999. IEEE Transactions in Biomedical Engineering 46(9):1065-1074.

TABLE 1
Summary of model parameters for unmyelinated model.

| ρₐₓₒₙ [7] | 1.1 Ωm |
|---|---|
| ρ_{ecf} [10] | 2.0 Ωm longitudinally |
| | 12.5 Ωm radially |
| Nernst potentials (calculated | Sodium 60 mV |
| using Nernst equation for | Potassium −90 mV |
| mammalian axons at 37° C.) | |
| Leakage Nernst potential [15] | −60 mV |
| Resting membrane potential | −70 mV |
| [15] | |
| Membrane specific capacitance | 1 μF/cm² |
| [15] | |

TABLE 2
Summary of model parameters for myelinated model.

| | | |
|---|---|---|
| Myelin segment length [16] | 300 | μm |
| g-ratio ([18] and see text) | 0.8 | |
| ρₐₓₒₙ [7] | 1.1 | Ωm |
| Nodal membrane specific | 2 | μF/cm² |
| capacitance [7] | | |
| ρ_{ecf} [10] | 2.0 | Ωm longitudinally |
| | 12.5 | Ωm radially |
| Sodium Nernst potential (see | 60 | mV |
| table 1) | | |
| Leakage Nernst potential [20] | −80.01 | mV |
| Resting membrane potential | −80 | mV |
| [20] | | |

TABLE 3
Conduction velocities and channel transit times.

| | | | | |
|---|---|---|---|---|
| | | External | Channel | |
| Axon diam. | Channel | velocity | velocity | Transit |
| [μm] | area [μm²] | [m/s] | [m/s] | time [ms] |

Unmyelinated axons

| | | | | |
|---|---|---|---|---|
| 0.5 | 1000 | 0.55 | 0.55 | 18 |
| 1.0 | 1000 | 0.73 | 0.72 | 14 |
| 2.0 | 500 | 1.00 | 0.98 | 10 |
| 2.0 | 1000 | 1.00 | 0.98 | 10 |
| 2.0 | 2000 | 1.00 | 0.98 | 10 |
| 2.0 | 5000 | 1.00 | 0.99 | 10 |
| 5.0 | 1000 | 1.53 | 1.51 | 6.6 |

Myelinated axons

| | | | | |
|---|---|---|---|---|
| 2.0 | 1000 | 21 | 20 | 0.49 |
| 5.0 | 1000 | 43 | 39 | 0.26 |
| 10 | 500 | 56 | 50 | 0.20 |
| 10 | 1000 | 56 | 51 | 0.20 |
| 10 | 2000 | 56 | 51 | 0.20 |
| 10 | 5000 | 56 | 54 | 0.19 |
| 20 | 1000 | 83 | 48 | 0.21 |

### APPENDIX

The unmyelinated model uses the standard Hodgkin-Huxley [14] equation:

$$J . n = {C}_{m} \frac{\partial {V}_{m}}{\partial t} + {g}_{Na} ( {V}_{m} - {E}_{Na} ) + {g}_{K} ( {V}_{m} - {E}_{K} ) + {g}_{L} ( {V}_{m} - {E}_{L} )$$

where: J.n is the normal outwardly-directed membrane current (there is no tangential component)
   Cₘ is the membrane capacitance per unit area
   E_{Na}, E_{K}, and E_{L} are respectively the sodium, potassium and leakage Nernst potentials (see table 1) and Vₘ the membrane potential
   g_{Na}, g_{K}, and g_{L} are respectively the sodium, potassium, and leakage membrane conductances per unit area

The leakage conductance g_{L} is a constant with a value of 3 S/m. g_{Na} and g_{K} are given by:

g_{Na}= g̅_̅{̅N̅a̅}̅m³h

g_{K}= g̅_̅{̅K̅}̅n⁴

where g̅_̅{̅N̅a̅}̅ and g̅_̅{̅K̅}̅ are constants having values of 1200 S/m and 360 S/m respectively.

The activation parameters m, h, and n are given by:

$$\frac{\partial m}{\partial t} = k \cdot ( {α}_{m} ( 1 - m ) - {β}_{m} m )$$

$$\frac{\partial h}{\partial t} = k \cdot ( {α}_{h} ( 1 - h ) - {β}_{h} h )$$

$$\frac{\partial n}{\partial t} = k \cdot ( {α}_{n} ( 1 - n ) - {β}_{n} n )$$

where k is a common acceleration factor for temperature compensation (see below), and the rate parameters αₘ, βₘ, αₕ, βₙ and βₙ are functions of Vₘ and the resting membrane potential Vᵣ given by (in SI units):

$${α}_{m} = \frac{2500 ( 1 - 40 ( {V}_{m} - {V}_{r} ) )}{exp ( 2.5 - 100 ( {V}_{m} - {V}_{r} ) ) - 1}$$

$${β}_{m} = 4000 exp ( - 500 ( {V}_{m} - {V}_{r} ) / 9 )$$

$${α}_{h} = 70 exp ( - 50 ( {V}_{m} - {V}_{r} ) )$$

$${β}_{h} = \frac{1000}{exp ( 3 - 100 ( {V}_{m} - {V}_{r} ) ) + 1}$$

$${α}_{n} = \frac{100 ( 1 - 100 ( {V}_{m} - {V}_{r} ) )}{exp ( 1 - 100 ( {V}_{m} - {V}_{r} ) ) - 1}$$

$${β}_{n} = 125 exp ( - 12.5 ( {V}_{m} - {V}_{r} ) )$$

The kinetics of m, h, and n are all found to speed up with increasing temperature, and the ‘acceleration factor’ k is included to reflect this. Rattay and Aberham provide a detailed discussion of k and we have used their value of 12.

The myelinated model uses Sweeney's [20] modification of Chiu's [19] 2-channel model featuring sodium and leakage conductances only. The main model equation is thus:

$$J . n = {C}_{m} \frac{\partial {V}_{m}}{\partial t} + {g}_{Na} ( {V}_{m} - {E}_{Na} ) + {g}_{L} ( {V}_{m} - {E}_{L} )$$

where g_{L} is a constant having the value 1280 S/m and g_{Na} is given by:

g_{Na}= g̅_̅{̅N̅a̅}̅m²h

where g̅_̅{̅N̅a̅}̅ is a constant having the value 14,450 S/m. (Note the quadratic dependence on m in this model rather than the cubic dependence in the Hodgkin-Huxley model). The activation parameters m and h are given by:

$$\frac{\partial m}{\partial t} = {α}_{m} ( 1 - m ) - {β}_{m} m$$

$$\frac{\partial h}{\partial t} = {α}_{h} ( 1 - h ) - {β}_{h} h$$

$$where \text{:}$$

$${α}_{m} = \frac{1000 ( 97 + 363 ( {V}_{m} - {V}_{r} ) )}{1 + exp ( 5.85 - 189 ( {V}_{m} - {V}_{r} ) )}$$

$${β}_{m} = \frac{{α}_{m}}{exp ( 240 ( {V}_{m} - {V}_{r} ) - 5.71 )}$$

$${α}_{h} = \frac{{β}_{h}}{exp ( 200 ( {V}_{m} - {V}_{r} ) - 1.1 )}$$

$${β}_{h} = \frac{15600}{1 + exp ( 2.4 - 100 ( {V}_{m} - {V}_{r} ) )}$$

All equations are expressed in SI units.

1. A neural interface comprising one or more electrode-coupled microchannels,
each electrode-coupled microchannel comprising;
a microchannel for accommodating regenerating nerve axons; and,
one or more electrodes exposed to the interior of said microchannel.
wherein the one or more electrodes comprise;
(i) a stimulation array suitable for generating an extracellular stimulus current which induces an action potential in an axon in the microchannel; and/or
(ii) a recording array for detecting an extracellular signal in the microchannel indicative of an action potential in an axon in the microchannel.

2. (canceled)

3. A neural interface according to claim 1 comprising two or more recording arrays which are axially spaced in the microchannel.

4. A neural interface according to claim 1 wherein the recording array comprises three electrodes connected in a tripole to an amplifier circuit.

5-6. (canceled)

7. A neural interface according to claim 1 wherein the recording array comprises a single electrode connected to a differential amplifier circuit.

8-20. (canceled)

21. A neural interface according to claim 1 wherein the stimulation array is suitable for generating an extracellular stimulus current which specifically induces an action potential in motor axons.

22. A neural interface according to claim 21 wherein the action potential induced in the axon is bi-directional.

23-25. (canceled)

26. A neural interface according to claim 21 wherein the action potential induced in the axon is uni-directional.

27. A neural interface according to claim 26 wherein the action potential is induced in the axon by a first stimulus current in a first region of the microchannel and a second stimulus current in a second region of the microchannel,
wherein the first and second regions are longitudinally spaced in the microchannel and the first stimulus current is greater than the second stimulus current,
such that the action potential is induced in the axon in the direction from the first region to the second region of the microchannel.

28-35. (canceled)

36. A neural interface according to claim 1 wherein the electrode coupled microchannel comprises a stimulation array and a recording array, said arrays being arranged in the microchannel such that an antidromic action potential is induced in the axon by the stimulation array in response to orthodromic action potentials detected in the axon by the recording array.

37-40. (canceled)

41. A neural interface according to claim 1 wherein the microchannels are formed from a biocompatible dielectric material.

42-47. (canceled)

48. A method of detecting an action potential in a nerve axon comprising;
providing a neural interface according to claim 1 comprising an electrode-coupled microchannel containing a nerve axon in a conductive medium, and;
detecting electrical signals in the medium which are indicative of an action potential in the nerve axon.

49-54. (canceled)

55. A method according to claim 48 comprising determining from said signals one or more of the amplitude, direction, velocity, frequency and pattern of the action potentials in the microchannel.

56. (canceled)

57. A method of stimulating an action potential in a nerve axon comprising;
providing a neural interface according to claim 1;
said interface comprising a microchannel containing a nerve axon in a conductive medium, and
one or more electrodes exposed to the interior of said microchannel, said one or more electrodes forming a stimulation array, and;
stimulating said stimulation array to produce an extracellular stimulus current in the conductive medium which induces an action potential in the nerve axon.

58. A method according to claim 57 wherein the action potential induced in the axon is bi-directional.

59. A method according to claim 57 wherein the action potential induced in the axon is uni-directional.

60. A method according to claim 59 wherein the action potential in the nerve axon is induced by generating a first stimulus current in a first region of the microchannel and a second stimulus current in a second region of the microchannel,
wherein the first and second regions of the microchannel are axially spaced and the first stimulus current is greater than the second stimulus current,
such that an action potential is induced in the nerve axon in the direction from the first to the second region.

61. A method according to claim 59 wherein the action potential in the nerve axon is induced by generating a current between a cathode located in the microchannel and grounded anodes outside the channel,
wherein the cathode is positioned closer to a proximal end of the microchannel than a distal end,
such that the first stimulus current between the cathode and the proximal end is greater than the second stimulus current between the cathode and the distal end of the microchannel and the action potential is induced in the axon in the direction from the proximal end to the distal end of the microchannel.

62. A method according to claim 59 wherein the action potential in the nerve axon is induced by;
generating a first stimulus current between a cathode and the first anode and a second stimulus current between the cathode and a second anode,
wherein the first and second anodes are axially spaced along the microchannel and the cathode positioned between the anodes and wherein the first stimulus current is greater than the second stimulus current
such that the action potential is induced in the axon in the direction from the first anode to the second anode of the microchannel.

63-64. (canceled)

65. A method of blocking an orthodromic action potential in a nerve axon comprising;
providing a neural interface according to claim 1, said interface comprising an electrode coupled microchannel containing a nerve axon in a conductive medium and a stimulation array comprising one or more electrodes contacting the conductive medium, and
generating a stimulus current in the microchannel through stimulation array to induce an antidromic action potential in the nerve axon,
said antidromic action potential blocking the passage of the orthodromic action potential in the axon.

66-79. (canceled)
